(19) United States
(12) Patent Application Publication
(10) Pub. No.: US 2007/0202097 A1
(43) Pub. Date: Aug. 30, 2007

(54) Monoclonal Antibodies That Recognise Mucosal Addressin Cell Adhesion Molecule-1 (Madcam-1), Soluble Madcam-1 And Uses Thereof

(76) Inventors: Geoffrey W. Krissansen, Auckland (NZ); Euphemia Leung, Auckland (NZ); Klaus B. Lehnert, Auckland (NZ)

Correspondence Address: EDWARDS ANGELL PALMER & DODGE LLP, P.O. BOX 55874, BOSTON, MA, 02205, US

(21) Appl. No.: 10/548,390

(22) PCT Filed: Mar. 10, 2004
(86) PCT No.: PCT/NZ04/00052

§ 371 Date: Apr. 18, 2007

(30) Foreign Application Priority Data
Mar. 10, 2003 (NZ) 524665
Mar. 24, 2003 (NZ) 524939

(51) Int. Cl. A61K 39/395; C12N 5/06; C07K 16/28; G01N 33/53

(52) U.S. Cl. 424/133.1; 435/7.92; 530/388.22; 435/334

(57) ABSTRACT

The present application describes novel monoclonal antibodies, fragments and derivatives thereof which recognise mucosal addressin cell adhesion molecule-1 (MAdCAM-1). Also described are methods which utilise the monoclonal antibodies. Further, a soluble form of MAdCAM-1 is described as well as methods which utilise its presence in a subject.

### FIELD

The present invention relates to novel antibodies, fragments and derivatives thereof, and uses therefor. In particular, the invention relates to monoclonal antibodies, fragments and derivatives thereof which recognise Mucosal addressin cell adhesion molecule-1 (MAdCAM-1), methods of diagnosis and monitoring of at least inflammation in a subject, and treatment of such inflammation. The invention also relates to a soluble form of Mucosal addressin cell adhesion molecule-1 (MAdCAM-1) and methods which utilise it.

### BACKGROUND

MAdCAM-1 is a cell-surface immunoglobulin (Ig) superfamily member composed of two extracellular Ig domains, followed by a mucin-like domain, a transmembrane domain, and a short cytoplasmic domain.^{1,2} It interacts via its N-terminal Ig domain with the lymphocyte homing receptor α4β7,³⁻⁵ which plays a critical role in forming the gut-associated lymphoid system.⁶ Antibodies against either the integrin α4β7 or MAdCAM-1 inhibit the homing of intestinal-seeking lymphocytes.^{7,8} MAdCAM-1 purified from mesenteric lymph nodes is decorated on its mucin domain with selectin-binding carbohydrate determinants that are able to support the rolling of lymphocytes under shear.⁹ MAdCAM-1 promotes the adhesion of T and B cells, monocytes/macrophages, and potentially eosinophils, basophils, and differentiated mast cells to the vascular endothelium.¹⁰

MAdCAM-1 RNA transcripts are predominantly expressed in the small intestine, mesenteric lymph nodes, colon, and spleen; and very weakly expressed in human pancreas and brain.^{1,2} Rodent MAdCAM-1 protein is detectable on endothelia in Peyer's patches, mesenteric lymph nodes, lamina propria of the small and large intestine,^{11,12} the lactating mammary gland,¹³ as well as on sinus-lining cells surrounding the periarteriolar lymphocyte sheath and follicle areas of the spleen.¹⁴ It is also displayed on follicular dendritic cells in Peyer's patches at sites associated with microenvironmental homing decisions, and on follicular dendritic cells in peripheral lymph nodes after primary immunization with antigen.¹⁵ Human MAdCAM-1 is widely expressed on endothelia in both lymphoid and non-lymphoid tissues from embryonic week 7 onwards, and only gradually becomes polarized to mucosal vessels after birth.¹⁶ It is found at similar sites as those described in rodents, and is strongly expressed on inflamed appendix, and moderately expressed in thymic medulla, and on high endothelial venules (HEV) of tonsils.¹⁷ It is expressed on nasal-associated lymphoid tissue (NALT) HEV and follicular dendritic cells.¹⁸ MAdCAM-1 is upregulated on HEV-like vessels in a variety of chronic inflammatory diseases, and may mediate increased leukocyte traffic into inflamed tissues. Thus, it can be found on HEV in the chronically inflamed pancreas of the non-obese diabetic mouse,¹⁹ on endothelia and choroid plexus epithelial cells in chronic relapsing experimental autoimmune encephalomyelitis (EAE),²⁰⁻²² on venules in the genital tract of mice infected with Chlamydia trachomatis,²³ and on gut lamina propria venules in inflammatory bowel disease.²

The upregulation of MAdCAM-1 on chronically inflamed endothelia, especially HEV, is in accord with the notion that this vascular addressin has a particular role to play in recruiting lymphocytes during the inflammatory process. In accord, it has been demonstrated to mediate the progression of Type I diabetes in the nonobese diabetic (NOD) mouse,²⁴ a chronic progressive form of EAE,²⁵ and inflammatory bowel disease.²⁶⁻²⁸ It supports mucosal lymphocyte adhesion to hepatic endothelium in chronic inflammatory liver disease.²⁹

Also, MAdCAM-1 is thought to be involved in lymphocyte recruitment in Helicobactor plyori-induced gastritis⁵². Duodenal ulcers are associated with the presence of Helicobacter plyori in the stomach.

Circulating soluble forms of certain cell adhesion molecules (CAMs) including ICAM-1, and VCAM-1 are elevated in inflammatory, infectious, and malignant diseases.³⁰ Levels of these soluble CAMs correlate with disease activity.

Bibliographic details of the publications referred to herein are collected at the end of the description.

### OBJECT

It is an object of the present invention to provide one or more of: novel antibodies, fragments and derivatives thereof, pharmaceutical compositions and kits comprising said antibodies or fragments or derivatives thereof, diagnostic methods, methods of treatment of inflammation, soluble MAdCAM-1, and methods of detecting and purifying same.

### STATEMENT OF INVENTION

In one aspect of the present invention there is provided a monoclonal antibody 17F5 or fragment or derivative thereof, wherein the antibody is produced by hybridoma cell line ATCC PTA-5010.

In another aspect the invention provides a monoclonal antibody 377D10 or fragment or derivative thereof, wherein the antibody is produced by hybridoma cell line ATCC PTA-5007.

In a further aspect, the invention provides a monoclonal antibody 314G8 or fragment or derivative thereof, wherein the antibody is produced by hybridoma cell line ATCC PTA-5008.

In yet a further aspect, the invention provides a monoclonal antibody 201F7 or fragment or derivative thereof, wherein the antibody is produced by hybridoma cell line ATCC PTA-5009.

In another aspect, the invention provides a monoclonal antibody 355G8 or fragment or derivative thereof, wherein the antibody is produced by hybridoma cell line ATCC PTA-5011.

In a further aspect, the invention provides a hybridoma cell line (ATCC PTA-5010) which produces a monoclonal antibody 17F5.

In another aspect, the invention provides a hybridoma cell line (ATCC PTA-5007) which produces a monoclonal antibody 377D10.

In a further aspect, the invention provides a hybridoma cell line (ATCC PTA-5008) which produces a monoclonal antibody 314G8.

In another aspect, the invention provides a hybridoma cell line (ATCC PTA-5009) which produces a monoclonal antibody 201F7.

In yet another aspect, the invention provides a hybridoma cell line (ATCC PTA-5011-which produces a monoclonal antibody 355G8

Antibodies derived from hybridoma cells 201F7, 314G8, 377D10 and 355G8 may be characterised by their recognition of the first 19 domain of MAdCAM-1. Antibody derived from hybridoma 17F5 may be characterised by its recognition of the mucin domain of MAdCAM-1.

In yet another aspect, there is provided a method of blocking the interaction of MAdCAM-1 with the integrin α4β7 the method comprising at least the step of bringing said MAdCAM-1 into contact with a monoclonal antibody or fragment or derivative thereof in accordance with the invention.

In a related broad aspect, the present invention provides a method of treating inflammation mediated by MAdCAM-1, the method comprising at least the step of administering to a subject in need thereof an effective amount of one or more of the monoclonal antibodies, or fragments or derivatives thereof, herein before described.

Preferably, the method is performed for the purposes of treating one or more of the following inflammatory disorders: appendicitis, pancreatitis, inflammatory bowel disease (including Crohn's disease and ulcerative colitis), inflammatory liver disease, diabetes, multiple sclerosis and other demyelinating diseases, duodenal ulcers, arthritis, asthma and other allergic inflammatory diseases, atherosclerosis, and inflammation associated with transplantation and chronic infection.

In another aspect of the invention there is provided a method of detecting the presence of MAdCAM-1 in a sample the method comprising at least the step of placing the sample in contact with one or more of the monoclonal antibodies, or fragments or derivatives thereof, as herein before described.

In yet another aspect, the invention provides a method of diagnosing or monitoring inflammation in a subject said method comprising at least the steps of:
Providing a sample from a subject; and
Determining the level of soluble MAdCAM-1 present in said sample.

Preferably, the sample from a subject is a biological fluid.

In another aspect of the invention there is provided a method of diagnosing or monitoring inflammation in a subject, the method comprising at least the steps of:
Providing at least one test sample from a subject;
Providing at least one monoclonal antibody, or fragment or derivative thereof, as herein before described;
Placing said at least one monoclonal antibody in contact with said test sample; and,
Determining the level of MAdCAM-1 in said test sample.

Preferably the method further comprises comparing the level of MAdCAM-1 in said test sample with a standard or base level.

Preferably, a method of this aspect of the invention employs ELISA. Alternatively, or additionally, the method employs one or more of RIA, immunoprecipitation, Western blotting, immunohistochemical staining, affinity chromatography, competitive binding assays, and agglutination assays.

In another aspect, the invention provides a pharmaceutical composition comprising at least one or more of the monoclonal antibodies or fragments or derivatives thereof as herein before described, in combination with one or more pharmaceutically acceptable diluents, carriers and/or excipients.

Further, the invention provides the use of one or more of the monoclonal antibodies or fragments or derivatives thereof as herein before described in the manufacture of a medicament for the treatment of inflammation in a subject.

In yet another aspect, the invention provides the use of a monoclonal antibody or fragment or derivative thereof as herein before described in a method for the purification of MAdCAM-1.

In yet a further aspect, the invention provides a kit for diagnosing or monitoring inflammation in a subject, the kit comprising at least one or more of the monoclonal antibodies or fragments or derivatives thereof as herein before described.

A kit according to this aspect of the invention may further comprise one or more control samples comprising a known level of MAdCAM-1 or a fragment or derivative thereof

In another aspect, the invention provides soluble MAdCAM-1 isolated from a human subject.

In a related aspect, the invention also provides the use of soluble MAdCAM-1, or a fragment or derivative thereof, in the manufacture of a kit for diagnosing or monitoring inflammation.

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

### FIGURES

These and other aspects of the present invention, which should be considered in all its novel aspects, will become apparent from the following description, which is given by way of example only, with reference to the accompanying figures, in which:

FIG. 1 Illustrates that anti-MAdCAM-1 mAbs specifically recognize human MAdCAM-1. (a) Anti-MAdCAM-1 mAbs recognize NIH-3T3 cells engineered to express human MAdCAM-1, but do not stain untransfected parental NIH 3T3 cells. NIH-3T3 cells stably transfected with a huMAdCAM-1 expression plasmid, and untransfected parental NIH-3T3 cells, were stained with the five anti-MAdCAM-1 mAbs. Results are shown for the 314G8 only, but identical results were obtained with the other four mAbs. (b) Four of the anti-MAdCAM-1 mAbs specifically recognize recombinant Fc fusion proteins possessing the first Ig domain of MAdCAM-1. Recombinant human Fc fusion proteins possessing either the first Ig or mucin domains of human MAdCAM-1, or complete extracellular domain of human ICAM-1, were resolved by SDS-PAGE, transferred to a nitrocellulose membrane, and screened with each of the anti-MAdCAM-1 mAbs, or with an anti-human-Fc antibody as control. MAbs 201F7, 314G8, 377D10 and 355G8 detected the 50 kDa human MAdCAM-1 Ig domain 1-Fc fusion protein, but not human ICAM-1-Fc (latter data only shown for 201F7 mAb). M, human MAdCAM-1 Ig domain-1-Fc fusion protein; I, human ICAM-1-Fc.

FIG. 2 Illustrates epitope mapping of anti-MAdCAM-1 mAbs. ELISA confirms that mAbs 201F7, 314G8, 377D10 and 355G8 recognize the first Ig domain of MAdCAM-1 (a), whereas mAb 17F5 recognizes the mucin domain (b). In (a), anti-MAdCAM-1 mAbs were immobilized on anti-mouse Fc antibody-coated wells, and tested for their ability to capture recombinant MAdCAM-1-Fc or MAdCAM-1 Ig domain 1-Fc in solution. Captured MAdCAM-1-Fc molecules were detected with a goat anti-human Fc antibody peroxidase conjugate and ABTC, and optical density at 405 nm recorded. In (b), fusion proteins possessing either the complete extracellular domain, or mucin domain of MAdCAM-1 were coated onto wells, and screened with each of the anti-MAdCAM-1 mAbs. Immunoreactivity was detected with an anti-mouse Fc antibody peroxidase conjugate, developed by incubation ABTS diammonium salt, and optical density at 405 nm recorded. (c) MAbs 201 F7, 314G8, and 377D10 recognize an identical or overlapping site in the N-terminal Ig domain of MAdCAM-1. MAdCAM-1-Fc coated wells were incubated with a biotinylated anti-MAdCAM-1 mAb in the presence of excess amounts of unlabelled competitor anti-MAdCAM-1 mAb. ELISAs were developed with extravdin-POD and ABTS diammonium salt, and optical density recorded.

FIG. 3 Illustrates that anti-MAdCAM-1 mAbs 201F7, 314G8, 377D10 and 355G8 block T cell adhesion to MAdCAM-1. (a) Human H9 T cells were tested for their ability to adhere to MAdCAM-1-Fc-coated wells that had previously been incubated with tissue culture supernatants containing anti-MAdCAM-1 mAbs, or with normal tissue culture medium as a control (Con). (b) Mouse TK-1 T cells were tested for their ability to adhere to human MAdCAM-1-Fc and VCAM-1-Fc-coated wells pretreated with anti-MAdCAM-1 mAbs as in (a). Cell binding to MAdCAM-1 was specific as cells failed to bind to wells coated with the FCS control substrate. The numbers of cells adhering are shown.

FIG. 4 Illustrates that anti-MAdCAM-1 mAbs stain venules in lymphoid organs, and inflamed tissue. The 314G8 (a,c), 201F7 (e), 377D10 (f), 355G8 (g), and 17F5 (h) mAbs specifically stain (brown) venules in osteoarthritic synovium, whereas a mouse ascites control did not stain (b,d). Sections were counterstained with hematoxylin. Magnification, a,b 40×; c-h, 100×. 314G8 mAb specifically stains venules in spleen, small intestine, and colon (malignant schwanoma). Serial sections of human spleen (i, j), small intestine (k, l), and colon (m,n) were stained for MAdCAM-1 (brown) using the 314G8 mAb (i,k,m), or mouse ascites control (j,l,n). Sections were counterstained with hematoxylin. Magnification, 40×.

FIG. 5 Illustrates a sandwich ELISA that detects sMAdCAM-1 in serum and urine. (a) The ability of different pairs of anti-MAdCAM-1 mAbs to capture and detect recombinant sMAdCAM-1. Recombinant sMAdCAM-1-Fc (70 ng) was captured and detected with various MAdCAM-1 antibody pairs. FCS was used as an antibody control. Illustrated is a representative experiment repeated in triplicate, with results expressed as optical density+SD. (b) A representative ELISA standard curve using recombinant sMAdCAM-1, and mAbs 377D10, and 355G8 as capture and detection mAbs, respectively. The standard curve was repeated in triplicate, and optical density+SD recorded. (c) Detection and quantification of sMAdCAM-1 in serum. MAbs 377D10 and 355G8 were used to capture and detect sMAdCAM-1 in the serum of 6 healthy donors. The related soluble CAMs ICAM-1-Fc and VCAM-1-Fc (1000 ng/mL, respectively), and FCS were included as antigen specificity controls. Illustrated is a representative experiment repeated in triplicate, with results expressed as optical density+SD. (d) Detection and quantification of sMAdCAM-1 in urine. MAbs 377D10 and 355G8 were used to capture and detect sMAdCAM-1 in the urine of 3 healthy donors (results of 2 donors shown in graph). A 1:50 dilution of mouse ascites coated onto plates was employed as a control capture antibody. FCS, BSA, and mouse urine were included as antigen specificity controls. Human serum (diluted 10×), and sMAdCAM-1-Fc (MAdCAM-1, 1000 ng/mL) were included for comparison.

### PREFERRED EMBODIMENT(S)

The following is a description of the preferred forms of the present invention given in general terms in relation to the application of the novel anti-human MAdCAM-1 monoclonal antibodies derived from the hybridoma cell lines designated 17F5, 355G8, 201F7, 314G8, and 377D10, and also a soluble form of MAdCAM-1, described for the first time by the inventors. The invention is further elucidated from the disclosure given under “Experimental Basis of the Invention” herein below, which provides specific examples thereof.

Deposits of the hybridoma cell lines designated 17F5, 355G8, 201F7, 314G8, and 377D10 have been provided in accordance with the Budapest Treaty on the international recognition of the deposit of micro-organisms for the purpose of patent procedure, to the American Type Culture Collection (ATCC), Manassas, Va., United States of America. Details of the deposits are as follows:

| Hybridoma | Deposit Designation | Deposit Date |
|---|---|---|
| 17F5 | PTA-5010 | 18 Feb. 2003 |
| 377D10 | PTA-5007 | 18 Feb. 2003 |
| 314G8 | PTA-5008 | 18 Feb. 2003 |
| 201F7 | PTA-5009 | 18 Feb. 2003 |
| 355G8 | PTA-5011 | 18 Feb. 2003 |

It should be noted that the designations 17F5, 355G8, 201F7, 314G8, and 377D10 may be used interchangeably herein to refer to either a hybridoma or the antibody derived therefrom. For example hybridoma 17F5 produces an antibody of the same name. Whether or not reference is made to the hybridoma or to the antibody derived therefrom should be apparent from the context in which the designation is used.

Mucosal addressin cell adhesion molecule (MAdCAM-1) is a key player in mediating the infiltration of leukocytes into chronically inflamed tissues. The inventors have identified five novel anti-MAdCAM-1 monoclonal antibodies (mAbs), designated 17F5, 355G8, 201F7, 314G8, and 377D10 generated by fusion of P3X63Ag8.653 myeloma cells with spleen cells from Balb/c mice immunized with recombinant human MAdCAM-1-Fc.

The antibodies of the invention can be characterised on the basis of isotype and epitope mapping and, in some cases, their ability to block binding of MAdCAM-1 to α4β7 molecules, as detailed herein after in the section headed “Results”. The inventors have identified that the latter four mAbs (above) recognize the ligand-binding first Ig domain of MAdCAM-1 and block T cell adhesion to MAdCAM-1. The blocking of T cell binding or adhesion to MAdCAM-1 has application in methods for treatmnent of inflammation in at least human subjects.

The mAb 17F5, which recognizes the mucin domain, does not block T cell adhesion to MAdCAM-1. Nonetheless, the inventors contemplate that this antibody, as with the mAbs 355G8, 201F7, 314G8, and 377D10, finds use in detecting-and purifying MAdCAM-1, including the newly identified soluble form of MAdCAM-1, as well as in methods for diagnosing and monitoring progression of inflammation in a subject as detailed herein after. MAb 17F5 might also find use in analysing the decoration of the MAdCAM-1 mucin domain by carbohydrate moieties.

Analysis by the inventors of a large panel of paraffin-embedded human tissues revealed that the 314G8 mAb detected MAdCAM-1 on venules in the spleen and small intestine, but in apparent disagreement with four previous studies, MAdCAM-1 was not detected in either the marginal zones of the splenic white pulp, or in the duodenum, or colon. It will be appreciated that such a result in no way detracts from the diagnostic and therapeutic aspects of the invention which may centre on increases, or relative increases, in levels of MAdCAM-1 (in cells, tissues or fluids for example) during episodes of inflammation.

MAdCAM-1 was strongly expressed in the synovium of osteoarthritis patients, predominantly on the endothelial lining of blood vessels, but also within the vessel lumen. An enzyme-linked immunosorbent assay (ELISA), based on mAbs 314G8 and 355G8, was developed and used to identify whether soluble MAdCAM-1 was present in body fluids. The assay surprisingly detected soluble MAdCAM-1 in the serum, and urine of healthy donors, at levels similar to those of soluble forms of the related CAMs ICAM-1 and VCAM-1. This is the first time a soluble form of MAdCAM-1 has been described. The inventors contemplate that the level of soluble MAdCAM-1 in subjects presenting inflammation, particularly chronic inflammatory diseases, may allow for diagnosis and monitoring of inflammation in such subjects, as may detection of levels of the membrane-bound form of MAdCAM-1. Measurement of soluble MAdCAM-1 is advantageous as collection of serum, plasma, or urine is relatively non-invasive.

As above mentioned, the hybridoma's of the invention were generated by fusion of P3X63Ag8.653 myeloma cells with spleen cells from Balb/c mice immunized with recombinant human MAdCAM-1-Fc. Details of the materials and methods used by the inventors are provided herein after under the heading “Materials and Methods”.

The hybridomas of the invention may be subcloned, grown and maintained using standard techniques in the art. For example, they may be grown and maintained in vitro in media such as DMEM or RPMI-1640. Alternatively, this may be done in vivo as ascites tumors in an animal of choice.

The antibodies of the invention may be isolated from culture supernatants, ascites fluid or serum using standard procedures known in the art to which the invention relates. An example of such techniques is provided herein after under the heading “Materials and Methods”. However, by way of example, isolation or purification may occur via one or more of chromatographic procedures such as affinity chromatography, Protein-A or G Sepharose or hydroxyapatite, and thiophilic gel chromatography, or via ammonium sulphate precipitation, centrifugation with liquid polymers, gel electrophoresis or dialysis.

Furthermore, inasmuch as an antibody is proteinaceous, the antibodies of the present invention may also be produced via standard recombinant techniques and as reviewed for example by Siegel (2002). The inventors consider recombinant techniques to be a preferable means of producing antibodies on a commercial scale for the therapeutic and diagnostic applications described herein after.

It should be understood that the present invention extends to fragments or derivatives of each of the monoclonal antibodies generated by the inventors, provided they exhibit at least a degree of the activity of the original intact monoclonal antibody, that is specificity for, or an ability to interact with or bind to, MAdCAM-1. It is not necessary, for the purposes of the invention that the fragment or derivative be capable of acting as an antibody; that is to say, the fragment or derivative need not be capable of recruiting immune system cells to the site of binding to MAdCAM-1 in vivo.

“Fragments” of the antibodies is intended to encompass a portion of one of the intact antibodies, generally the antigen binding or variable region of the antibody. Examples of antibody fragments include Fab, Fab′ F(ab′)₂, and Fv fragments. Those of ordinary skill in the art to which the invention relates will recognise methods to generate such antibody fragments from the antibodies 17F5, 355G8, 201F7, 314G8, and 377D10 of the invention. However, by way of general example proteolytic digestions of intact antibodies may be used, or the fragments may be directly produced via recombinant nucleic acid technology.

As used herein, “derivatives” of a monoclonal antibody of the invention includes, for example, hybrid and recombinant versions of 17F5, 355G8, 201F7, 314G8, and 377D10. “Hybrid” and “recombinant” versions of the antibodies include, for example, humanised antibodies, diabodies, triabodies, and single chain antibodies.

“Humanised” antibodies are essentially hybrid or chimeric antibodies containing domains derived from human sources and domains derived from the animal in which an antibody may have been generated. In the present case, they are mouse/human-hybrid antibodies. Humanised antibodies in accordance with the invention will generally comprise the mouse CDR (complementarity determining region or antigen binding site) of one of 17F5, 355G8, 201F7, 314G8, and 377D10 fused to appropriate human antibody domains or regions necessary to form a functional antibody, for example. Humanization of murine antibodies can be achieved using techniques known in the art, for example by epitope-guided selection (Wang et al, 2000). The methods of Jones et al (1986), or Maynard and Georgiou (2000) provide further examples.

Humanisation of antibodies may help reduce the immunogenicity of the antibodies of the invention in humans for example. Reduced immunogenicity can be obtained by transplanting murine CDR regions to a homologous human β sheet framework (termed CDR grafting; refer to Riechmann et al, and Jones et al 1986).

Those of skill in the art to which the invention relates will appreciate the terms “diabodies” and “triabodies”. These are molecules which comprise a heavy chain variable domain (VH) connected to a light chain variable domain (VL) by a short peptide linker that is too short to allow pairing between the two domains on the same chain. This promotes pairing with the complementary domains of one or more other chain encouraging the formation of dimeric or trimeric molecules with two or more functional antigen binding sites. The resulting antibody molecules may be monospecific or multispecific (eg bispecific in the case of diabodies). Such antibody molecules may be created from two or more of the antibodies of the present invention using methodology standard in the art to which the invention relates; for example, as described by Holliger et al (1993), and Tomlinson and Holliger (2000).

Additionally, as may be useful for certain applications, monoclonal antibodies of the invention may be modified by labelling with a compound which provides a detectable signal; for example, enzymes, fluorescent agents and radioisotopes. Those of general skill in the art to which the invention relates will readily identify such suitable labelling systems. However, examples of suitable labelling systems and protocols are provided herein after under the heading “Materials and Methods”.

Further, the antibodies of the invention may be immobilised on a solid phase, for example to aid in purification of MAdCAM-1 or for diagnostic purposes or for therapeutic purposes.

Additionally, the antibodies of the invention may be used as carriers, for example to carry toxins, radionucleotides, isotopes, genes, or other therapeutic molecules to cells or tissues to aid in therapy.

As mentioned herein before, antibodies of the invention may be produced via standard recombinant procedures. Accordingly, the present invention also extends to nucleic acids encoding an antibody, fragment or derivative thereof, constructs comprising same, and host cells comprising said constructs. Nucleic acids in accordance with the invention may be DNA, RNA or cDNA for example, double stranded or single stranded, sense or antisense.

Nucleic acids encoding the antibodies may be readily identified on the basis of the amino acid sequence of the antibodies, the genetic code, and the understood degeneracy therein. Nucleic acids encoding the antibodies of the invention may be isolated from the hybridoma cells and subsequently characterised using procedures standard in the art. For example, a nucleic acid probe may be designed based on the amino acid sequence of a portion of an antibody and then used to isolate genes encoding the heavy and/or light chains of the murine antibodies. Alternatively, nucleic acids may be generated by standard chemical synthesis methodology (for example using phosphoramidite and solid phase chemistry).

It will be appreciated that the amino acid sequence of an antibody of the invention may be determined using standard methodology; for example, the technique of Edman degradation and HPLC or mass spectroscopy analysis (Hunkapiller et al, 1983), may be used.

It should be understood that a nucleic acid in accordance with the invention, is an “isolated” or “purified” nucleic acid. An “isolated” or “purified” nucleic is one which has been identified and separated from the environment in which it naturally resides, for example within the hybridoma. It should be appreciated that ‘isolated’ does not reflect the extent to which the nucleic has been purified or separated from the environment in which it naturally resides.

Isolated nucleic acids of the invention may be readily spliced into appropriate cloning and/or expression vectors to form constructs for ultimate recombinant expression of the antibodies 17F5, 355G8, 201F7, 314G8, and 377D10 or fragments or derivatives thereof.

Nucleic acid constructs in accordance with the invention will generally contain heterologous nucleic acid sequences; that is nucleic acid sequences that are not naturally found adjacent to the nucleic acid sequences of the invention. The constructs or vectors may be either RNA or DNA, either prokaryotic or eukaryotic, and typically are viruses or a plasmid. Suitable constructs are preferably adapted to deliver a nucleic acid of the invention into a host cell and are capable of replicating in such cell. Recombinant constructs comprising nucleic acids of the invention may be used, for example, in the cloning, sequencing, and expression of nucleic acid sequences of the invention.

Those of skill in the art to which the invention relates will recognise many constructs suitable for use in the present invention.

A recombinant construct or vector comprising a nucleic acid molecule of the invention may be generated via recombinant techniques readily known to those of ordinary skill in the art to which the invention relates.

In the case of expression constructs, the inventors contemplate the use in the present invention of vectors containing regulatory sequences such as promoters, operators, repressors, enhancers, termination sequences, origins of replication, and other appropriate regulatory sequences as are known in the art. Further, the vectors may contain secretory sequences to enable an expressed protein to be secreted from its host cell. In addition, the expression vectors may contain fusion sequences (such as those that encode a heterologous amino acid motif, for example hexahistidine, glutathione S-transferase, ubiquitin) which lead to the expression of inserted nucleic acid sequences of the invention as fusion proteins or peptides.

In accordance with the invention, transformation of a construct into a host cell can be accomplished by any method by which a nucleic acid sequence can be inserted into a cell. For example, transformation techniques include transfection, electroporation, microinjection, lipofection, adsorption, and biolistic bombardment.

As will be appreciated, transformed nucleic acid sequences of the invention may remain extrachromosomal or can integrate into one or more sites within a chromosome of a host cell in such a manner that their ability to be expressed is retained.

Any number of host cells and transgenic expression systems known in the art may be utilised in cloning and expressing nucleic acid sequences of the invention. Host cells may be prokaryotic or eukaryotic. Examples of suitable host cells include Chinese Hamster Ovary (CHO) cells, NSO cells, insect cells, yeast, and E coli. Transgenic organisms include mouse, chickens, goats, and plants (tobacco, maize, soy, rice, and wheat).

A recombinant antibody in accordance with the invention may be recovered from a transformed host cell, or culture media, or transgenic organism following expression thereof using a variety of techniques standard in the art. For example, detergent extraction, osmotic shock treatment and inclusion body purification. The antibody may be further purified using techniques such as affinity chromatography, ion exchange chromatography, filtration, electrophoresis, hydrophobic interaction chromatography, gel filtration chromatography, Protein-A or -G Sepharose, and chromatofocusing.

It should be appreciated that inasmuch as the invention also extends to fragments and derivatives of the monoclonal antibodies specifically referred to herein, nucleic acid encoding the antibodies may be appropriately modified. For example, the coding sequence for heavy- and light-chain constant domains may be replaced with an homologous human domain. Alternatively, the mouse CDR regions may be transplanted to a homologous human beta sheet framework. In this way, antibody derivatives, such as humanised antibodies may be generated via recombinant techniques.

Antibodies produced in accordance with the invention may find use, for example, as research tools, diagnostic and therapeutic agents.

The inventors believe that the monoclonal antibodies or fragments or derivatives thereof described herein find application in the regulation of MAdCAM-1-mediated physiological functions. Particularly, the inventors have shown that the antibodies 201F7, 314G8, 377D10 and 355G8 block T cell interaction with MAdCAM-1 and therefore may be applicable to the treatment of inflammation in a subject.

In one broad embodiment the invention provides a method of blocking the interaction of MAdCAM-1 with the integrin α4β7, or more broadly MAdCAM-1 with leukocytes, the method comprising at least the step of bringing said MAdCAM-1 into contact with a monoclonal antibody or fragment or derivative thereof in accordance with the invention. This method may be conducted in vivo or in vitro.

In a related embodiment, the method relates to the treatment of inflammation. As used herein, the term “treatment” is to be considered in its broadest context. The term does not necessarily imply that a subject is treated until total recovery.

Accordingly, “treatment” broadly includes the modulation or control of inflammation mediated by MAdCAM-1, aberrant or otherwise, amelioration of the symptoms or severity of an inflammatory disorder mediated by MAdCAM-1, or preventing or otherwise reducing the risk of developing an inflammatory disorder mediated by MAdCAM-1. The inventors contemplate the invention being particularly applicable to treatment of chronic inflammation.

The term “inflammatory disorder(s)” should be taken to mean any undesired physiological condition which involves inflammation. Examples of inflammatory disorders applicable to the present invention include: appendicitis, pancreatitis, inflammatory bowel disease (including Crohn's disease and ulcerative colitis), inflammatory liver disease, diabetes, multiple sclerosis and other demyelinating diseases, duodenal ulcers, arthritis, asthma and other allergic inflammatory diseases, atherosclerosis, and inflammation associated with transplantation and chronic infection.

A “subject” as used herein is preferably a human.

In as much as the present invention relates to the treatment or modulation of inflammation it also provides a pharmaceutical composition comprising an antibody of the invention or a derivative or fragment thereof in association or combination with one or more pharmaceutically acceptable diluents, carriers and/or excipients.

As-used herein, the phrase “pharmaceutically acceptable diluents, carriers and/or excipients” is intended to include substances that are useful in preparing a pharmaceutical composition, may be co-administered with an antibody of the invention, or derivative or fragment thereof, while allowing same to perform its intended function, and are generally safe, non-toxic and neither biologically nor otherwise undesirable. Examples of pharmaceutically acceptable diluents, carriers and/or excipients include solutions, solvents, dispersion media, delay agents, emulsions and the like.

Those skilled in the art of protein-based treatments will readily appreciate a variety of pharmaceutically acceptable diluents, carriers and/or excipients which may be employed in compositions of the invention. By way of example, suitable liquid carriers, especially for injectable solutions, include water, aqueous saline solution, aqueous dextrose solution, and the like, with isotonic solutions being preferred for intravenous, intraspinal, and intracistemal administration. Methods of formulating suitable compositions may be found in references such as Gennaro A R: Remington: The Science and Practice of Pharmacy, 20th ed., Lippincott, Williams & Wilkins, 2000.

In addition to standard diluents, carriers and/or excipients, a pharmaceutical composition comprising an antibody, derivative or fragment thereof in accordance with the invention may be formulated with additional constituents, or in such a manner, so as to enhance the activity of the antibody, derivative or fragment thereof, or help protect the integrity of same. For example, the composition may further comprise constituents which provide protection against proteolytic degradation, or decrease antigenicity of the antibody, or derivative or fragment thereof, upon administration to a subject. For example, compounds of this invention may also be PEGylated to increase their lifetime in vivo.

Additionally, it is contemplated that a pharmaceutical composition in accordance with the invention may be formulated with additional active ingredients which may be of benefit to a subject in particular instances. For example, therapeutic agents that target the same or different facets of the disease process may be used.

As will be appreciated by those of ordinary skill in the art to which the invention relates, the antibodies, or fragments or derivatives thereof and carriers, diluents or excipients of a composition of the invention may be converted to customary dosage forms such as solutions, orally administrable liquids, injectable liquids, tablets, coated tablets, capsules, pills, granules, suppositories, trans-dermal patches, suspensions, emulsions, sustained release formulations, gels, aerosols, powders and immunoliposomes. Additionally, sustained release formulations may be utilised. The dosage form chosen will reflect the mode of administration desired to be used. Particularly preferred dosage forms include orally administrable tablets, gels, pills, capsules, semisolids, powders, sustained release formulation, suspensions, elixirs, aerosols, ointments or solutions for topical administration, injectable liquids, or any other appropriate dosage forms.

The inventors contemplate the antibodies (or fragments or derivatives thereof of this embodiment of the invention being present within a pharmaceutical composition in an amount suitable to deliver a desired amount to a subject.

The present invention also pertains to methods for the modulation or treatment of inflammation comprising at least the step of administering to a subject in need thereof a therapeutically effective amount of an antibody of the invention, or fragment or derivative thereof, or a pharmaceutical composition comprising same.

As used herein, a “therapeutically effective amount”, or an “effective amount” is an amount necessary to at least partly attain a desired response. A person of ordinary skill in the art will be able without undue experimentation, having regard to that skill and this disclosure, to determine a therapeutically effective amount of a compound of this invention for a given disease or injury.

The inventors contemplate administration of an antibody, fragment or derivative thereof, or pharmaceutical composition of the invention by any means capable of delivering such to a target site within or on the body of a subject; a “target site” is a site at which an inflammatory event has, or is predicted to, occur, or a site which may otherwise benefit from the delivery of said antibody, derivative or fragment thereof. It should be understood that the site of administration may be distinct from the target site. In general, compounds of this invention will be administered as pharmaceutical compositions by one of the following routes: oral, topical, systemic (eg. transdermal, intranasal, or by suppository), parenteral (eg. intramuscular, subcutaneous, or intravenous injection), by administration to the CNS (eg. by intraspinal or intracisternal injection); by implantation, and by infusion through such devices as osmotic pumps, transdermal patches, and the like. Skilled persons may identify other appropriate administration routes.

As will be appreciated, the dose of an antibody, fragment or derivative thereof or composition administered, the period of administration, and the general administration regime may differ between subjects depending on such variables as the severity of symptoms of a subject, the type of disorder to be treated, the mode of administration chosen, and the age, sex and/or general health of a subject. However, by way of general example, the inventors contemplate administration of from approximately 30 μg to 300 mg per kilogram (mg/Kg) mass of the animal, for example, 0.3 to 30 mg/Kg, with lower doses such as 0.003 to 0.3 mg/Kg, e.g. about 0.03 mg/Kg, being appropriate for administration through the cerebrospinal fluid, such as by intracerebroventricular administration, and higher doses such as 3 to 300 mg/Kg, e.g. about 30 mg/Kg, being appropriate for administration by methods such as oral, systemic (eg. transdermal), or parenteral (e.g. intravenous) administration.

It should be appreciated that administration may include a single daily dose or administration of a number of discrete divided doses as may be appropriate.

Further example procedures relating to preparation and administration of the antibodies (and fragments or derivatives thereof) of the invention will be appreciated by persons of ordinary skill in the art to which the invention relates.

The inventors contemplate that the presence of soluble MAdCAM-1, in a sample taken from a subject may be indicative of inflammation, or at least a chronic inflammatory event, presenting in said subject. Accordingly, the present invention also extends to methods of diagnosing or monitoring inflammation in a subject said method comprising at least the steps of 1) providing a sample from a subject to be tested; and, 2) determining the level of soluble MAdCAM-1 present in said sample.

It will be appreciated that such method may also be applicable to monitoring the effectiveness of any treatment administered to a subject.

In another embodiment, the invention provides a general method of detecting the presence of MAdCAM-1 in a sample by placing the sample in contact with a monoclonal antibody or fragment or derivative thereof in accordance with the invention.

Related to the above embodiment is the applicability of the antibodies or fragments or derivatives thereof to a method of diagnosing or monitoring inflammation. A method of this embodiment generally comprises at least the steps of: 1) providing at least one test sample from a subject; 2) providing at least one monoclonal antibody or fragment or derivative thereof in accordance with the invention; 3) placing said at least one antibody or fragment or derivative thereof in contact with said test sample; and 4) determining the level of MAdCAM-1 in said test sample.

A test sample from a subject may include any fluid or tissue sample. However, serum, plasma, and urine are preferred samples.

The inventors contemplate the sample being used in the form from which it is derived from the subject. However, the sample is preferably processed to increase the specificity of the subsequent assay to be conducted. Processing steps may differ depending on the technique to be used to assess interaction of an antibody or fragment or derivative thereof with MAdCAM-1 within a sample. However, examples of processing steps include dilution of the sample in a suitable diluent, for example a buffer solution such as PBS, or a protein containing buffer solution such as 0.5% BSA (bovine serum albumen) in PBS. As it will be appreciated, such dilution steps may be used to decrease the viscosity of the sample and/or to decrease background non-specific binding.

Methods of detecting MAdCAM-1 or of monitoring or diagnosing inflammation may include at least one control sample. These may include positive and negative controls having either a known level of MAdCAM-1 or fragment or derivative thereof, in the case of a positive control, or no MAdCAM-1 in the case of a negative control. Persons of ordinary skill in the art to which the invention relates will appreciate examples of positive and negative controls suitable for use in the present invention from a reading of the present description and that provided under the heading “Examples”, although they may also readily appreciate alternatives.

Further, such controls may also include a sample taken from a subject being tested, wherein said control sample was taken at a different time (ie a time during which the subject is known not to be presenting an inflammatory event, or alternatively during a time when the subject was known to be presenting a chronic inflammatory event).

As used herein, the term “MAdCAM-1 or fragment or derivative thereof”, unless otherwise stated, should be taken in its broadest possible context. It may include for example MAdCAM-1 (in its soluble form or otherwise) purified from natural sources, purified recombinant MAdCAM-1, soluble fragments of MAdCAM-1 which may represent for example the ligand binding first Ig domain or the mucin domain, or fusion proteins comprising MAdCAM-1 or a fragment thereof. Additionally, the MAdCAM-1 or a fragment or derivative thereof may be modified by incorporation of a detectable label or the like. In one embodiment of the invention, the MAdCAM-1 may be presented on the surface of primary cells or recombinant cells, or cell lines. Further examples of MAdCAM-1, fragments and derivatives thereof which are applicable to the present invention are provided herein after under the heading “Examples”.

A method of detecting MAdCAM-1 or of monitoring or diagnosing inflammation may employ one or more known immunological techniques such as enzyme linked immunosorbent assay (ELISA) (sandwich ELISA, double sandwich ELISA, direct ELISA, microparticle ELISA), radioimmunoassay (RIA), immunoprecipitation, Western blotting, immunohistochemical staining, or agglutination assay. Protocols for carrying out such techniques are readily available; for example, see “Antibodies a Laboratory Manual”, Cold Spring Harbor Laboratory Press (1988), or the protocols described herein after under the heading “Materials and Methods”.

Further a detection or diagnostic method may include the use of affinity chromatography wherein an antibody of the invention or a fragment or derivative thereof is immobilised on an appropriate solid support. Skilled persons will readily recognise standard methodology for carrying out this aspect of the invention.

Alternatively, standard competitive binding assays may be employed.

Preferably a method of detection or diagnosing or monitoring in accordance with the invention uses ELISA. As mentioned above, ELISA may be conducted according to standard methodology in the art to which the invention relates. However, ELISA parameters of use in the present invention are exemplified herein after under the heading “Examples”.

It will be appreciated that ELISA may incorporate both direct and indirect detection means, and that an antibody of the invention, or fragment or derivative thereof, may be used as either capture or detection antibodies. As will be appreciated, one or more of the antibodies of the invention, or fragments or derivatives thereof, may be used in a single assay; for example where two antibodies of the invention do not recognise the same antigenic determinant on MAdCAM-1, one antibody may be used as capture antibody and a distinct antibody of the invention used as detection antibody. Examples of this are elucidated herein after where it is demonstrated that either of the antibodies 17F5 and 233G8 may be used as capture antibodies in combination with any one of the antibodies 201F7, 314G8, and 377D10 as detection antibodies. Alternatively, an antibody distinct from those of the present invention may be used in an assay as capture antibody or detection antibody; examples of the combination of an antibody of the invention with distinct antibodies in an ELISA are provided hereinafter. Further examples include mouse anti-human MAdCAM-1 mAb MCA2096Z (Serotec, Oxford, UK).

As will be appreciated by persons of ordinary skill in the art to which the invention relates the detection antibody used in an ELISA may be conjugated to a detectable label as herein before described.

Information of use in diagnosing or generally monitoring the inflammation status of a subject may be gained by making a direct comparison of the level of MAdCAM-1 in a test sample, with that of a determined base level or standard. For example, the inventors have identified that the average serum level of soluble MAdCAM-1 of a normal subject (ie a subject known not to present inflammation or more particularly a chronic inflammatory event) is within the range of approximately 180 ng/ml to approximately 314 ng/ml. In-the case-of soluble MAdCAM-1 present in urine the inventors have identified a range of from approximately 20 ng/ml to approximately 123 ng/ml within normal subjects. These concentrations may be used as base levels, with a result above this range being indicative of inflammation or chronic inflammatory disease. Preferably, the level necessary to be indicative of inflammation is a statistically significant increase of those ranges identified herein. However, even where there is no statistically significant increase, results obtained may provide valuable information about the inflammatory status of a subject.

It should be appreciated that the normal ranges of soluble MAdCAM-1 provided above are mere examples. Where samples are obtained from alternative body fluids and tissues, or where membrane-bound MAdCAM-1 is utilised, for example, the normal range may fall outside those quoted.

In accordance with the above it should be appreciated diagnosis or general determination of a subjects inflammatory status may be made by comparing the level of MAdCAM-1 present in a test sample against a database of results obtained from a range of other subjects.

Instead of utilising a standard or base level concentration of MAdCAM-1 (based on results obtained from a number of normal subjects) the base level concentration may be one which was determined from a single subject during a period when they were known not to present an inflammatory event, or during a period of chronic inflammation. This may be particularly applicable to cases of ongoing and/or intermittent inflammatory events or disorders where constant monitoring of the subjects status is required. For example, a base level may be determined during a period of remission from the disorder and the diagnostic procedure carried out at various times thereafter to assess inflammatory status. This may provide valuable information pertaining to progression of a disorder, or help in assessing whether treatment of the inflammation is proving successful.

In another embodiment the invention relates to a kit for monitoring or diagnosing: inflammation in a subject. The kit of this embodiment comprises at least one or more of the monoclonal antibodies of the invention or fragments or derivatives thereof. The kit may further comprise one-or more control samples comprising a known level of MAdCAM-1 or a fragment or derivative thereof.

As will be appreciated, antibodies or fragments or derivatives thereof in accordance with the invention may be used for the general purposes of detection and purification of MAdCAM-1, including the soluble form of MAdCAM-1. Such MAdCAM-1 may be from a natural or artificial source (such as a cell culture). Preferably, the MAdCAM-1 is of human origin. Those of ordinary skill in the art to which the invention relates will appreciate techniques by which this may be done. However, by way of example, affinity chromatography using antibodies, fragments or derivatives immobilised on a chromatographic support may be used (for example refer to Berlin et al. 1993).

As mentioned above the inventors have been the first to identify and describe a soluble form of MAdCAM-1 (which may be referred to herein as ‘soluble MAdCAM-1”). Accordingly, the present invention also provides such MAdCAM-1 in a form isolated from its natural environment, preferably from humans, more preferably from human urine or serum.

“Isolated” or “purified” soluble MAdCAM-1 is one which has been identified and separated from the environment in which it naturally resides. The terms isolated and purified are not intended to reflect the extent to which the soluble MAdCAM-1 has been purified or separated.

Soluble MAdCAM-1 may be isolated or purified from its natural environment in accordance with techniques described herein, and also having regard to methodology known in the art.

Isolated soluble MAdCAM-1 may find various applications. For example, it may be used as a control in a diagnostic assay or kit previously described herein. Alternatively, it may be used to generate further antibodies of therapeutic or diagnostic use. It may also be used as a substrate to study the process of cell adhesion, and other inflammatory events.

The invention will now be further elucidated by reference to the following non-limiting examples.

### EXAMPLES

Materials and Methods

Tissues

Human tissue sections were purchased from Imgenex (San Diego, Calif., USA), and osteoarthritis (OA) paraffin blocks were obtained from Dr Patrick McNeil (University of New South Wales, Australia).

Cell Culture

The mouse NIH3T3 fibroblast, TK1 thymic lymphoma, P3X63Ag8.653 myeloma, and human A375 and C2 melanomas, HeLa and DU145 epithelial carcinomas, and CFPac1 epithelial, HT29 epithelial, HUVEC umbilical vein vascular endothelial, kidney epithelial 293T, and H9 T cell lines were purchased from the American Type Culture Collection (ATCC; Rockville, Md., USA). Human HMEC endothelial cells were kindly provided by Dr Edwin Ades, National Centre for Infectious Disease, Atlanta, Ga. TK-1 T, and NIH 3T3 cells were grown in DMEM supplemented with 10% heat-inactivated fetal calf serum (FCS) (Invitrogen, Life Technologies, Auckland, New Zealand), 50 units/mL benzyl-penicillin, 50 μg/mL streptomycin, 2 mM L-glutamine (PSG), and 50 μM 2-mercaptoethanol (2-ME). A375 and 293T cells were cultured in the same media, but without 2-ME. P3X63Ag8.653 and H9 cells were cultured in RPMI-1640, 20% FCS, and PSG. C32, HeLa, and DU145 cells were grown in MEM with PSG and Earle's BSS supplemented with 1.5 g/L sodium bicarbonate, 0.1 mM non-essential amino acids, 1 mM sodium pyruvate and 10% FCS. HMEC cells were cultured in MCDB131 supplemented with 10 ng/mL of endothelial growth factor, 1 μg/mL hydrocortisone, and 10% FCS; HUVEC cells were grown in Ham's F12K medium with 2 mM L-glutamine supplemented with 1.5 g/L sodium bicarbonate, 0.1 mg/mL heparin and 30 to 50 μg/mL endothelial cell growth supplement (ECGS), and 10% FCS. CFPac1 cells were cultured in Iscove's modified Dulbecco medium with 10% FCS, and HT29 cells were grown in McCoy's 5A with 1.5 mM L-glutamine, and 10% FCS.

Production and Biotinylation of Anti-human MAdCAM-1 mAbs

Two female Balb/c mice were immunised with recombinant soluble MAdCAM-1-Fc fusion protein secreted from CHO cells³¹ according to standard procedures.³² Splenocytes from the immunised mice were fused separately with murine P3X63Ag8.653 myeloma cells, and hybridoma cells were selected in medium supplemented with HAT as described.³² Hybridoma cells secreting antibody recognising the immunogen in ELISA were cloned by two rounds of limited dilution. The hybridoma cell line 355G8 was derived from the splenocytes of the first mouse, while the hybridoma cell lines 17F5, 201F7, 314G8, 377D10 were derived from the second mouse. For production of tissue culture supernatants, the 17F5, 201F7, 314G8, 377D10 and 355G8 hybridoma cells were cultured at high density in RPMI 1640 supplemented with 5% heat-inactivated FCS. Ascites fluid was produced in nude mice according to standard procedures. Monoclonal antibodies were purified from ascites fluid by protein G-Sepharose (Sigma) affinity chromatography using standard procedures.³² Purified mAbs were biotinylated with biotin-NHS-succinimide ester (BiotinTag Micro Biotinylation Kit, Sigma).

Production of Recombinant Soluble IgCAMs, and Cell Transfection

Complementary DNA encoding human MAdCAM-1 has been described previously.^{1,2} DNA encoding the first Ig domain (amino acid residues 1 to 112) of human MAdCAM-1 was ligated to the C3 Fc fragment of pIG-I/IL-11RI3C (kindly provided by Dr. Keith Hudson, Genesis R&D, Auckland, NZ), and subsequently cloned into pcDNA3 to produce a construct (MAdCAM-1-D1-Fc) encoding a soluble form of the first Ig domain of MAdCAM-1. The sequence encoding the mucin domain (aa residues 226 to 319) of human MAdCAM-1 fused to a hexa-histidine tag was cloned into pET32C-C3-TCR5 (kindly donated by Ries Langley, University of Auckland) and transformed into Escherichia coli BL21(DE3) cells to produce an isolated form of the MAdCAM-1 mucin domain (MAdCAM-1-Muc-His). Recombinant MAdCAM-1-Muc-His protein was purified by nickel chelate chromatography (Qiagen). Complementary DNAs encoding the extracellular regions of human VCAM-1, ICAM-1 and MAdCAM-1 fused to the hinge, CH2 and CH3 domains of human IgG1, and inserted into the pEE14 expression vector, and stably transfected into CHO-K1 cells, have been described previously.³¹ Fc-fusion proteins were purified on immobilized protein A (PerSeptive Biosystems, Cambridge, Mass.). NIH 3T3 and 293T cells were stably transfected with MAdCAM-1 expression plasmids using Lipofectamine 2000 (Invitrogen, Life Technologies, Auckland, New Zealand). Briefly, cells were grown to confluence in 24 well plates, and each well was transfected with 3 μL of Lipofectamine 2000 complexed with 2 μg of plasmid DNA.

Antibodies, and Cytokines

Mouse ascites control antibody was purchased from Sigma, St. Louis, Mo., USA. All antibodies were titrated before use to determine appropriate dilutions for immunoperoxidase, immunoalkaline phosphatase, and immunofluorescence staining. Recombinant murine TNF-α was purchased from R&D Systems Inc, Abingdon, UK.

ELISA and Isotype Analysis

Recombinant soluble CAMs (70 μL of 10 μg/mL antigen) were coated onto MAXISORP plates overnight at 4° C. Different dilutions of each MAdCAM-1 mAb were added to wells in triplicate, and immunoreactivity detected by incubation with the appropriate secondary antibody and anti-biotin EctrAvidin peroxidase conjugate (Sigma; 1:1,000 dilution), or goat anti-human Fc peroxidase conjugate (1:10,000 dilution; Sigma) followed by development with ABTS (2′,2′-azino-bis-(3-ethylbenzthiazoline-6-sulfonic acid diammonium salt) (Roche, Basel, Switzerland). Alternatively, anti-MAdCAM-1 mAbs contained in tissue culture supernatants were immobilized on anti-mouse Fc antibody (100 μl 1:2000 dilution)-coated wells, and tested for their ability to capture recombinant MAdCAM-1-Fc (1 μg) or MAdCAM-1 Ig domain 1-Fc in tissue culture supernatant. Captured MAdCAM-1-Fc molecules were detected with a goat anti-human Fc antibody peroxidase conjugate and ABTC. Optical densities at 405 nm were recorded following immune detection. For antibody cross-blocking analysis, wells were coated with 100 μL of 10 μg/mL MAdCAM-1-Fc overnight at 4° C. Biotinylated anti-MAdCAM-1 mAbs (40 ng) were incubated in coated wells for 1 h at RT in the presence of 200 ng of unlabelled competitor anti-MAdCAM-1 mAb. Immunoreactivity was detected with ExtrAvidin-peroxidase conjugate, and developed by incubation with ABTS. Anti-MAdCAM-1 mAbs were isotyped with an IsoStrip kit (Boehringer Mannheim, Germany).

Sandwich ELISA for Soluble MAdCAM-1

In order to quantitate soluble human MAdCAM-1 (sMAdCAM-1) in cell culture supernatants, sera and plasma, purified anti-MAdCAM-1 capture mAbs in PBS were pre-coated (10 μg/mL; 100 μL/well) onto MAXISORP plates overnight at 4° C. The plates were washed thrice with PBS containing 0.2% Tween-20, and blocked with 3% (w/v) BSA in PBS. Sera (100 μl of 10 to 20× dilution), urine (100 μl, undiluted), or recombinant MAdCAM-1-Fc (70 ng) were incubated in blocked wells together with an excess of biotinylated anti-MAdCAM-1 detecting antibodies (10 μg/mL; 100 uL volume) at 4° C. overnight. Plates were washed thrice with PBS containing 0.2% Tween-20, and immunoreactivity detected by incubation with ExtrAvidin peroxidase conjugate for 30 min at room temperature (RT), followed by development with ABTS.

Immunofluorescence Staining and Immunohistochemistry

For immunofluorescence staining, cells were fixed with 4% paraformaldehyde in PBS for 20 min at RT, and were incubated for 30 min at RT with either specific primary mAbs, or with an isotypematched control mAb. Cells were then labelled for 20 min with FITC-conjugated secondary Abs, mounted in PBS/glycerol (Citifluor), and photographed. Single-colour immunohistochemistry was performed on paraffin sections using the Vectastain ABC kit (Vector Laboratories, Inc., Burlingame, Calif.). Paraffin sections were deparaffinized, and incubated with 3% hydrogen peroxide for 30 min at RT to block endogenous peroxidase activity. Sections were treated with an avidin/biotin blocking reagent (Vector Laboratories, Inc., Burlingame, Calif.), and incubated for 1 h at RT with primary anti-MAdCAM-1 mAbs diluted at 1:200. Bound antibodies were detected by the ABC method (Vectastain ABC Elite kit, Vector Laboratories), using diaminobenzidine tetrahydrochloride (Sigma Fast DAB, Sigma, Mo.) with metal enhancer tablets. The sections were counterstained with hematoxylin, dehydrated, and coverslipped in Hystomount (Hughes & Hughes Ltd, Wellington, Somerset). Mouse ascites (Sigma, Mo.) was used as a primary antibody control.

Adhesion Assay

Adhesion assays were performed as previously described.⁴ Briefly, Lab-Tek 16-well slides (Nalgene Nunc International, Rochester, N.Y.) were coated overnight at 4° C. with 10 μg/mL MAdCAM-Fc (70 μL/well) in PBS, and then blocked for 2 h with 3% BSA. For antibody blocking experiments, undiluted hybridoma culture supernatants, or tissue culture medium control, were added to MAdCAM-1-Fc-coated slides for 30 min at RT prior to addition of cells. TK-1 and H9 cells were activated by suspension in DMEM supplemented with 10% FCS, and 2 mmol/L each of Ca2+ and Mn2+, and added to coated slides at 1×10⁶ cells/well. For all assays, the slides were rinsed gently in PBS before fixing the cells in 2% glutaraldehyde. Cells were enumerated by light microscopy.

SDS-PAGE and Western Blot Analysis

For epitope mapping, soluble recombinant MAdCAM-1-Fc molecules were recovered from the culture supernatants (1 mL) of transfectants by selection with 20 μL of protein A Sepharose beads. They were subjected to SDS-PAGE under reducing conditions, and electrophoretically transferred to Hybond C Extra nitrocellulose membranes (Amersham Life Science, Amersham, UK). The membranes were blocked with 3% BSA in TBS (20 mM Tris pH7.6, containing 137 mM NaCl) for 1 h at RT, and then incubated for 60 min at RT in TBS containing 100 μg/mL BSA, and anti-MAdCAM-1 ascites diluted 1:500. Immunoreactivity was detected by incubating the membranes with horseradish peroxidase-conjugated sheep anti-mouse IgG (Sigma, Mo.) diluted 1:7,000, and development by enhanced chemiluminescence (Amersham International, Buckingham, UK) and autoradiography.

Results

Isolation of a Panel of Anti-human MAdCAM-1 mAbs

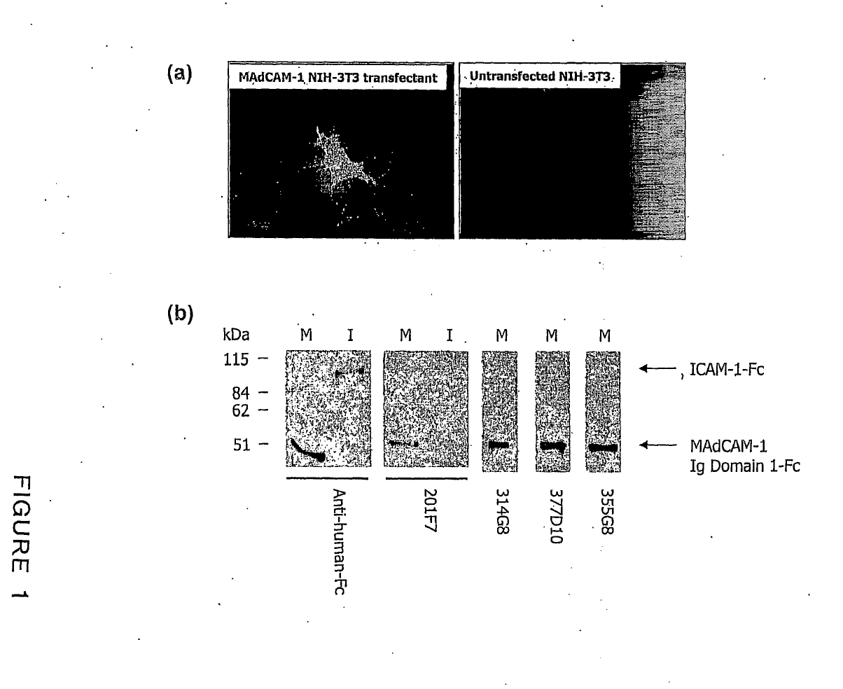

A panel of anti-human MAdCAM-1 mAbs designated 17F5, 201F7, 314G8, 377D10 and 355G8 was generated using conventional mAb production technologies (Methods). Hybridoma culture supernatants for each of the latter mAbs stained NIH-3T3 cells stably transfected with a huMAdCAM-1/pcDNA3 construct, but not parental NIH-3T3 cells, thereby confirming that each mAb specifically recognized MAdCAM-1 (FIG. 1a).

Isotyping and Epitope Analysis of Anti-human MAdCAM-1 mAbs

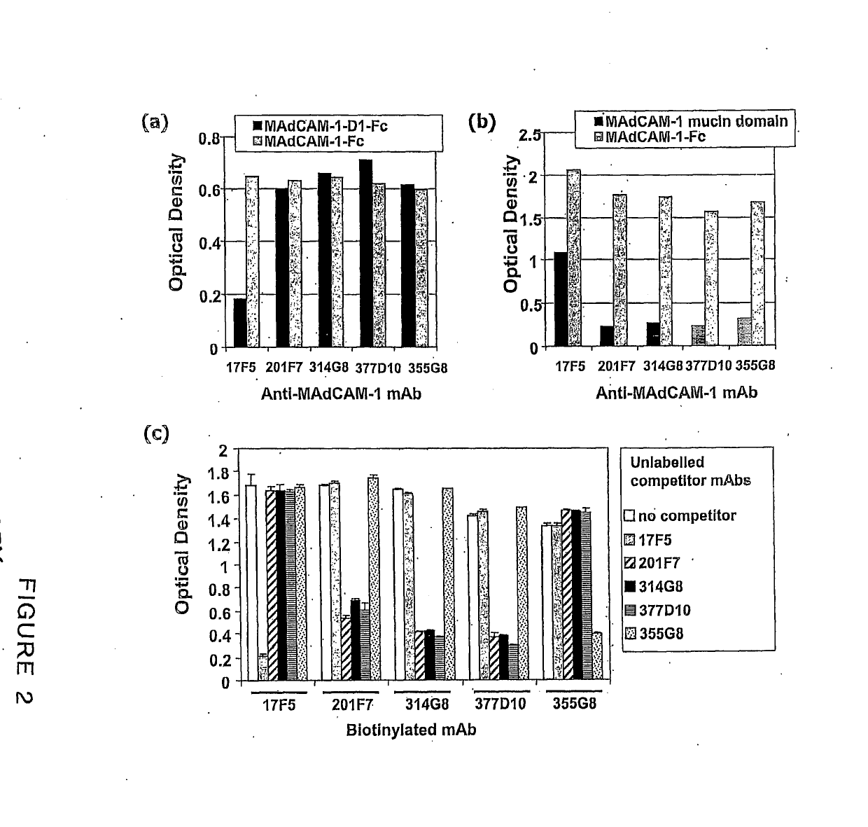

Each of the 17F5, 201F7, 314G8, 377D10 and 355G8 mAbs was typed as IgG1(kappa) (data not shown), and screened by Western blot (FIG. 1b), and ELISA (FIG. 2a,b) analysis for their ability to capture (FIG. 2a) and recognize (FIG. 2b) full-length MAdCAM-1, and either its first Ig domain produced as recombinant molecules in 293T cells, or an E. coli-derived recombinant MAdCAM-1 mucin domain. Epitope mapping indicated that epitopes recognized by the 201F7, 314G8, 377D10 and 355G8 mAbs mapped to Ig domain 1, whereas 17F5 recognized the mucin domain. Cross-blocking analysis revealed that mAbs 201F7, 314G8, and 377D10 recognize an identical or overlapping site in the N-terminal Ig domain of MAdCAM-1 (FIG. 2c).

Anti-MAdCAM-1 mAbs that Recognize Ig Domain 1 Block T Cell Attachment to MAdCAM-1

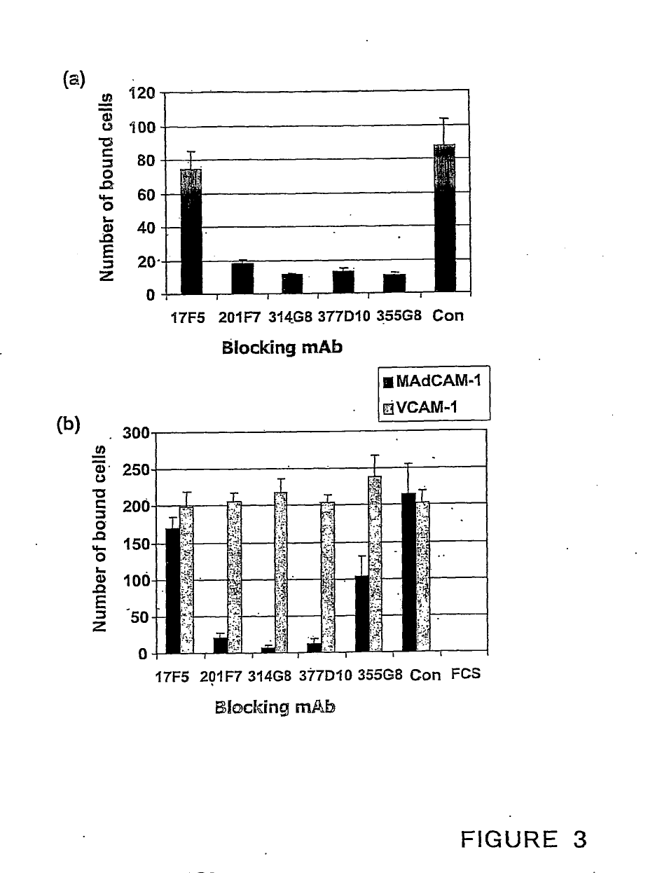

Each anti-MAdCAM-1 mAb was tested for its ability to block the binding of α4β7-expressing T cell lines TK-1 and H9 to recombinant human MAdCAM-1-Fc. Tissue culture supernatants from hybridomas expressing the 201F7, 314G8, 377D10 and 355G8 mAbs blocked both human H9 (FIG. 3a) and mouse TK-1 (FIG. 3b) cell binding to MAdCAM-1-Fc, whereas the 17F5 mAb that recognizes the mucin domain was a non-blocking mAb (FIG. 3). The 355G8 mAb was less effective at inhibiting the binding of mouse TK-1 cells versus human H9 cells to MAdCAM-1, revealing a potential difference in the structures of human and mouse α4β7. None of the blocking mAbs was able to inhibit T cell adhesion to VCAM-1, confirming their specificity for MAdCAM-1.

Employment of the Panel of Anti-MAdCAM-1 mAbs to Analyse the Distribution of Human MAdCAM-1

Initially, a panel of cell lines including HUVEC and HMEC endothelial cells; DU145, HT-29, CFPac-1 and HeLa epithelial cells; and A375 and C32 melanoma cells was examined by immunofluorescence staining for surface expression of MAdCAM-1. None of the latter cell lines was stained by any of the anti-MAdCAM-1 mAbs, regardless of whether the cells had previously been stimulated with TNF-α (data not shown).

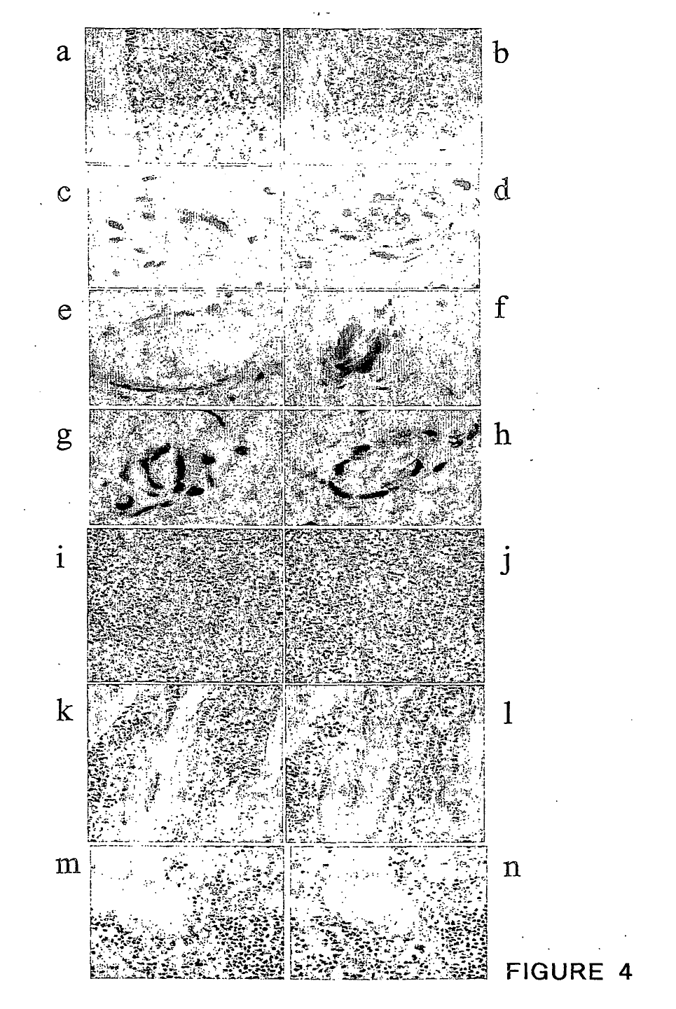

All five anti-MAdCAM-1 mAbs detected MAdCAM-1 in the synovium of osteoarthritis patients (FIG. 4a and h), predominantly on the endothelial lining of blood vessels, but also within the lumen of larger vessels (FIG. 4a). The latter result suggests a circulating soluble form of MAdCAM-1 exists in patients suffering from inflammatory disease.

The 314G8 mAb was used to examine the expression of MAdCAM-1 in a panel of paraffin-embedded human tissues contained in BA1 Human slides purchased from Imgenex. In accord with previously published results, MAdCAM-1 was expressed on venules in the spleen and small intestine (FIG. 4i to l and Table 1). No staining was observed either in the marginal zones of the splenic white pulp, or in the colon (data not shown). MAdCAM-1 was not detected in normal skin, breast, skeletal muscle, lung, liver, gallbladder, pancreas, stomach, rectum, kidney (cortex and medulla), bladder, prostate, uterus, placenta, umbilical cord, or brain. It was also not expressed in a variety of cancers including lung (squamous cell carcinoma and bronchioloalveolar carcinoma), salivary gland (adenoid cystic carcinoma), liver (hepatocellular carcinoma; metastatic adenocarcinoma from bile duct), stomach (adenocarcinoma; stromal sarcoma), rectum (adenocarcinoma), kidney (renal cell carcinoma), bladder (poorly differentiated transitional cell carcinoma), uterine cervix (squamous cell carcinoma), uterus (endometrial adenocarcinoma), adrenal gland (pheochromocytoma), ovary (metastatic adenocarcinoma from colon), or breast (infiltrating duct carcinoma). It was expressed on a few endothelia in venules of the colon of a patient with a malignant schwannoma (FIG. 4m,n). MAdCAM-1 was not detected in various tumour tissues contained in BB3 HistoArrays from Imgenex.

A MAdCAM-1 Immunoassay Assay Detects Soluble MAdCAM-1 in Body Fluids

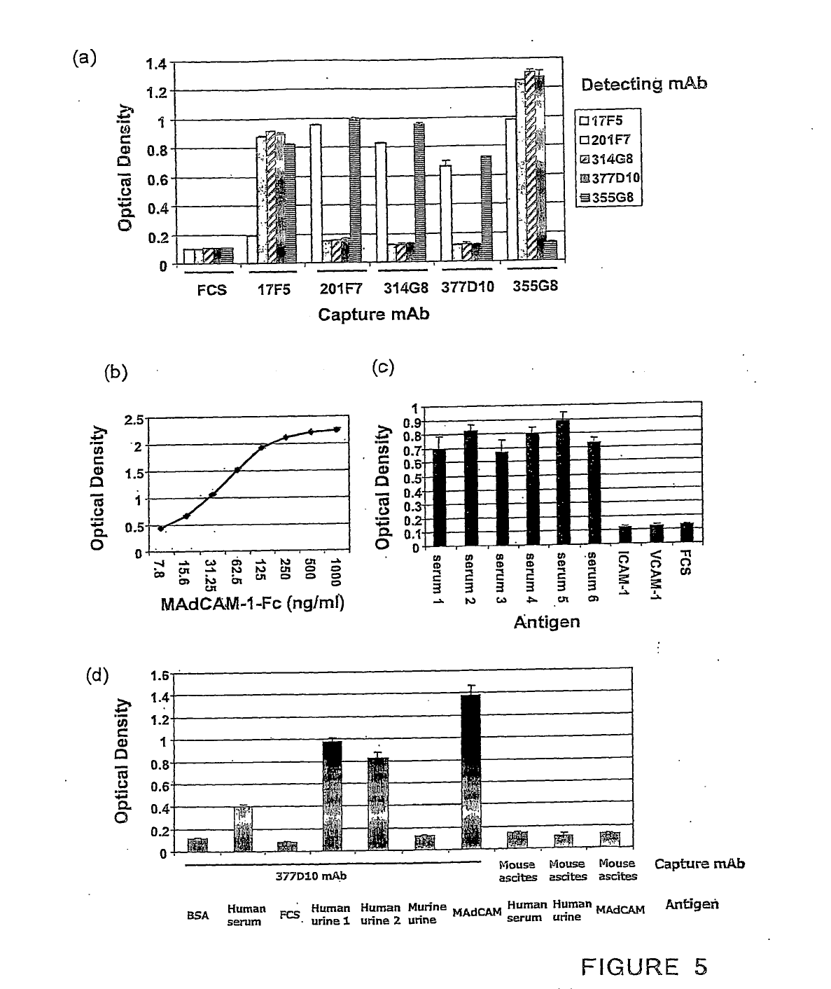

A sandwich ELISA was used to quantitate the levels of soluble MAdCAM-1 (sMAdCAM-1) in the sera and urine of healthy donors. Once again, a cross-blocking experiment demonstrated that the blocking mAbs 201F7, 314G8, and 377D10 recognized a common site in MAdCAM-1, and therefore would not be effective pairs for a sandwich ELISA (FIG. 5a). However, both the 17F5 and 355G8 mAbs worked well as capture mAbs with each of the biotinylated 201F7, 314G8, and 377D10 blocking mAbs being used as sMAdCAM-1 detection reagents. Vice versa, the 201F7, 314G8, and 377D10 mAbs also worked well as capture mAbs with each of the biotinylated 17F5 and 355G8 mAbs being used as sMAdCAM-1 detection reagents. The 377D10 and 355G8 mAbs were employed as capture and detection mAbs, respectively, for the remainder of the experiments. The ELISA was reasonably sensitive, being able to detect 0.78 ng of recombinant sMAdCAM-1 (FIG. 5b).

The MAdCAM-1 immunoassay was applied to the detection of sMAdCAM-1 in sera and urine sMAdCAM-1 was detected in the sera of 6 healthy donors at 236.5+55.8 ng/mL (FIG. 5c). Recombinant ICAM-1-Fc and VCAM-1-Fc, and FCS which were included as antigen controls to measure non-specific binding, generated only background levels of binding, demonstrating once again that the mAbs were specific for human MAdCAM-1sMAdCAM-1 was detected in the urine of healthy donors at 20-123 ng/mL, where mouse urine was included to measure background binding given that none of the anti-human MAdCAM-1 mAbs recognized mouse MAdCAM-1 (data not shown) (FIG. 5d). Mouse ascites were used as controls in place of the capture antibody, and generated only a background signal. BSA and FCS which were included as antigen controls to measure non-specific binding, and likewise generated only background levels of binding. Human serum (diluted 10×), and sMAdCAM-1-Fc were included for comparison as positive controls.

TABLE 1
Human cell lines and tissues tested for expression of
MAdCAM-1 antigen

Antibody

| Tissues | 17F5 | 201F7 | 314G8 | 377D10 | 355G8 |
|---|---|---|---|---|---|
| OA synovium | + | + | + | + | + |
| BA1 Human test slide | | | | | |
| Small intestine | ND | ND | + | ND | ND |
| Spleen | ND | ND | + | ND | ND |
| All remaining organs and | ND | ND | − | ND | ND |
| tumors | | | | | |

Discussion

The present work led to the development of five new anti-human MAdCAM-1 mAbs, four of which recognize the ligand-binding first Ig domain, and block the interaction of MAdCAM-1 with α4β7, and one of which specifically recognizes the MAdCAM-1 mucin domain. The 314G8 mAb, which recognizes MAdCAM-1 in paraffin-embedded human tissues, recognized venules in the spleen and small intestine. However, in apparent disagreement with three previous studies, the inventors did not detect MAdCAM-1 in either the marginal zones of the splenic white pulp, the duodenum, or normal colon. Veres et al.³³ detected MAdCAM-1 on structures in the lamina propria of the duodenal (region of gut separating the stomach from the small intestine) mucosa, but the structures identified were not specified. They were also rare, which may explain why the inventors did not detect them. Souza et al.,³⁴ and Briskin et al.¹⁷ have reported that MAdCAM-1 is expressed on vascular endothelial cells in the colonic lamina propria, and that expression is increased in patients with inflammatory bowel disease. Steiniger et al. reported that fibroblasts with an unusual phenotype located in the perifollicular zone, the outer marginal zone, and the T call zone of the splenic white pulp, stain for MAdCAM-1. Each of the latter studies were carried out with the two anti-MAdCAM-1 mAbs 10A6 and 10G3 developed by Briskin et al. 17 that detect MAdCAM-1 in cryosections but poorly or not at all in paraffin-embedded sections. The peculiar expression of MAdCAM-1 on fibroblasts, which are not known to express MAdCAM-1, might suggest that these mAbs cross-react with an unrelated determinant. The epitope recognized by the 314G8 mAb may be labile in the spleen, duodenum, and colon, but this seems unlikely given that the 314G8 mAb recognizes MAdCAM-1 in paraffin-embedded sections. Another possible explanation is that MAdCAM-1 is alternatively spliced at specific anatomic sites, such that different functional domains are represented. In such a scenario, MAdCAM-1 in the colon, duodenum, and on fibroblast-like splenic cells may represent differentially spliced forms that lack the first Ig domain of MAdCAM-1 and hence are not recognised by the 314G8 mAb. Another explanation is that regions that express MAdCAM-1 are rare in the commercial sections studied by the inventors. In a more extensive search the inventors found that the 314G8 mAb stained MAdCAM-1 on venules in the colon from a patient with malignant schwannoma.

MAdCAM-1 was very strongly expressed in the synovium of osteoarthritis patients, and was detected at this site by all five anti-MAdCAM-1 mAbs. This is in contrast with the study by Briskin et al. ¹⁷ using their MAdCAM-1 mAbs 10A6 and 10G3, which did not detect MAdCAM-1 in rheumatoid arthritis synovium. The inventors detected MAdCAM-1 within the vessel lumen of osteoarthritic synovium, indicating that circulating soluble forms of MAdCAM-1 exist in patients suffering from inflammatory disease. Increased levels of circulating soluble adhesion molecules have been proposed to be the key to understanding the prognosis and pathology of certain diseases (reviewed by Gearing and Newman³⁰). Indeed, many reports have shown that circulating soluble forms of Ig CAMs including ICAM-1, and VCAM-1 are elevated in inflammatory, infectious, and malignant diseases, whereas in contrast there is no previous description of soluble forms of MAdCAM-1. Thus, plasma concentrations of soluble ICAM-1 and VCAM-1 are increased in patients with atherosclerosis, ischemic heart disease, hyperlipidemia, diabetes, hyerglycemia, neurodegenerative disease including multiple sclerosis, rheumatoid arthritis, localized scleroderma, systemic lupus erythematosus, asthma, interstitial lung disease, graft rejection, various cancers, acute pyelonephritis, viral infection including HIV, dengue infections, spontaneous bacterial peritonitis, meningococcal disease, and cytomegalovirus infection among others. Levels of these soluble CAMs, which are regarded as surrogate markers of cell-surface expression, correlate with disease activity, and may be useful immunological markers to monitor the progression of some diseases, and the results of therapy. Some soluble CAMs are thought to be released from the cell-surface by proteolytic cleavage, while others are secreted as alternatively spliced forms lacking a transmembrane domain, or are anchored to the cell-surface by a glycosylphosphatidylinositol tail that could be released by phosphatidylinositol-specific phospholipase C.^{36, 37} Normal values of circulating soluble ICAM-1 and VCAM-1 as compiled by Gearing and Newman³⁰ were 245 ng/mL (average value from eight separate studies; range 102 to 450 ng/mL), and 459 ng/mL (average value from four separate studies; range 431 to 504 ng/mL), respectively. Subsequent studies have found similar levels, where circulating levels of soluble ICAM-1 are consistently half those of soluble VCAM-1.

This work is the first to detect soluble forms of MAdCAM-1 in biological fluids. The average serum level of MAdCAM-1 detected was 237 ng/mL (range 180 to 314 ng/mL), which is most similar to that of levels of ICAM-1 in serum. Raised concentrations of urinary soluble ICAM-1 have previously been reported in patients with bladder carcinoma,³⁸ kidney disease and renal allograft rejection,³⁹ and pyelonephritis.⁴⁰ The origins of urinary IgCAMs is unknown, but most likely include glomerular filtration, and shedding from proximal tubular epithelial cells in the case of ICAM-1.³⁹ The measurement of urinary soluble CAMs provides a noninvasive method to monitor disease progression. Normal average values of urinary soluble ICAM-1 and VCAM-1 range from 0.7 to 2.7 ng/mL, and 27 ng/mL, respectively. Here the inventors have demonstrated that soluble MAdCAM-1 is excreted into urine to give concentrations ranging from 20-123 ng/mL. For future quantitative comparisons urinary sMAdCAM-1 may be expressed as a ratio of urinary sMAdCAM-1/creatine (ng/mmol).

In summary, the inventors have revealed that MAdCAM-1 is secreted as a soluble form into serum and urine, and probably other biological fluids. As MAdCAM-1 is a marker of HEV which are induced at chronically inflamed sites, soluble MAdCAM-1 should be useful in monitoring and managing the progression of a variety of at least major chronic inflammatory diseases with which it is linked, either alone or in combination with other soluble adhesion markers. Further, the anti-MAdCAM-1 antibodies and assays of the invention may be useful therapeutically in the treatment of inflammation in humans. Similarly, they may be useful diagnostically to monitor presence and levels of MAdCAM-1.

The invention has been described herein with reference to certain preferred embodiments, in order to enable the reader to practice the invention without undue experimentation. However, a person having ordinary-skill in the art will readily recognise that many of the components and parameters may be varied or modified to a certain extent without departing from the scope of the invention. Furthermore, titles, headings, or the like are provided to enhance the reader's comprehension of this document, and should not be read as limiting the scope of the present invention.

The entire disclosures of all applications, patents and publications, cited above and below, if any, are hereby incorporated by reference.

The reference to any prior art in this specification is not, and should not be taken as, an acknowledgment or any form of suggestion that that prior art forms part of the common general knowledge in any country of the world.

Throughout this specification and any claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

### BIBLIOGRAPHY

1 Leung E, Greene J, Ni J, Raymond L G, Lehnert K, Langley R, Krissansen G W. Cloning of the mucosal addressin MAdCAM-1 from human brain: identification of novel alternatively spliced transcripts. Immunol. Cell. Biol. 1996; 74: 490-6.
2 Shyjan A M, Bertagnolli M, Kenney C J, Briskin M J. Human mucosal addressin cell adhesion molecule-1 (MAdCAM-1) demonstrates structural and functional similarities to the alpha 4 beta 7-integrin binding domains of murine MAdCAM-1, but extreme divergence of mucin-like sequences. J Immunol. 1996; 156: 2851-7.
3 Berlin C, Berg. E L, Briskin -M J, Andrew D P, Kilshaw P J, Holzmann B, Weissman I L, Hamann A, Butcher E C. Alpha 4 beta 7 integrin mediates lymphocyte binding to the mucosal vascular addressin MAdCAM-1. Cell 1993; 74:185-5.
4 Yang Y, Sammar M, Harrison J E, Lehnert K, Print C G, Leung E, Prestidge R, Krissansen G W. Construction and adhesive properties of a soluble MadCAM-1-Fc chimera expressed in a baculovirus system: phylogenetic conservation of receptor-ligand interaction. Scand J Immunol. 1995; 42: 235-47.
5 Briskin M J, Rott L, Butcher E C. Structural requirements for mucosal vascular addressin binding to its lymphocyte receptor α4β7. J. Immunol. 1996; 156: 719-26.
6 Wagner N, Lohler J, Kunkel E J, Ley K, Leung E, Krissansen G, Rajewsky K, Muller W. Critical role for beta7 integrins in formation of the gut-associated lymphoid tissue. Nature 1996; 382: 366-70.
7 Hamann A, Andrew D P, Jablonski-Westrich D, Holzmann B, Butcher E C. Role of α4-integrins in lymphocyte homing to mucosal tissues in vivo. J. Immunol. 1994; 152: 3282-93.
8 Palecanda A, Marshall J S, Li X, Briskin M J, Issekutz T B. Selective antibody blockade of lymphocyte migration to mucosal sites and mast cell adhesion. J. Leuk. Biol. 1999; 65: 649-57.
9 Berg E L, McEvoy L M, Berlin C, Bargatze R F, Butcher E C. L-selectin-mediated lymphocyte rolling on MAdCAM-1. Nature 1993; 366: 695-98.
10 Krissansen, G. W., Leung, E., and Lehnert, K. In: Guidebook to the extracellular matrix and adhesion proteins. T. E. Kreis, and R. Vale eds. Second edition. Oxford University Press, 1999.
11 Streeter P R, Berg E L, Rouse B T, Bargatze R F, Butcher E C. A tissue-specific endothelial cell molecule involved in lymphocyte homing. Nature 1988; 331: 41-6.
12 Picker L J, Butcher E C. Physiological and molecular mechanisms of lymphocyte homing. Annu. Rev. Immunol. 1992; 10: 561-91.
13 Van der Feltz M J M, de Groot N, Bayley J P, Lee S H, Verbeet M P H, de Boer H A. Lymphocyte homing and Ig secretion in the murine mammary gland. Scand. J. Immunol. 2001; 54: 292-300.
14 Kraal G, Schornagel K, Streeter P R, Holzmann B, Butcher E C. Expression of the mucosal vascular addressin, MAdCAM-1, on sinus-lining cells in the spleen. Am J Pathol. 1995; 147: 763-71.
15 Szabo M C, Butcher E, McEvoy L M. Specialization of mucosal follicular dendritic cells revealed by mucosal addressin-cell adhesion molecule-1 display. J. Immunol. 1997; 158: 5584-88.
16 Salmi M, Alanen K, Grenman S, Briskin M, Butcher E C, Jalkanen S. Immune cell trafficking in uterus and early life is dominated by the mucosal addressin MAdCAM-1 in humans. Gasteroenterol. 2001; 121: 853-64.
17 Briskin M, Winsor-Hines D, Shyjan A, Cochran N, Bloom S, Wilson J, McEvoy L M, Butcher E C, Kassam N, Mackay C R, Newman W, Ringler D J. Human mucosal addressin cell adhesion molecule-1 is preferentially expressed in intestinal tract and associated lymphoid tissue. Am. J. Pathol. 1997; 151: 97-110.
18 Csencsits K L, Jutila M A, Pascual D W. Nasal-associated lymphoid tissue: phenotypic and functional evidence for the primary role of peripheral node addressin in naive lymphocyte adhesion to high endothelial venules in a mucosal site. J. Immunol. 1999; 163: 1382-89.
19 Hanninen A, Taylor C, Streeter P R, Stark L S, Sarte J M, Shizuru J A, Simell O, Michie S A. Vascular addressins are induced on islet vessels during insulitis in nonobese diabetic mice and are involved in lymphoid cell binding to islet endothelium. J Clin Invest. 1993; 92: 2509-15.
20 O'Neill J K, Bufter C, Baker D, Gschmeissner S E, Kraal, G, Butcher E C, Turk J L. Expression of vascular addressins and ICAM-1 by endothelial cells in the spinal cord during chronic relapsing experimental allergic encephalomyelitis in the Biozzi AB/H mouse. Immunology. 1991; 72: 520-5.
21 Cannella B, Cross A H, Raine C S. Relapsing autoimmune demyelination: A role for vascular addressins. J. Neuroimmunol. 1991; 35: 295-300.
22 Steffen B J, Breier G, Butcher E C, Schulz M, Engelhardt B. ICAM-1, VCAM-1, and MAdCAM-1 are expressed on choroid plexus epithelium but not endothelium and mediate binding of lymphocytes in vitro. Am. J. Pathol. 1996; 148: 1819-38.
23 Kelly K A, Rank R G. Identification of homing receptors that mediate the recruitment of CD4 T cells to the genital tract following intravaginal infection with Chlamydia trachomatis. Infect. Immun. 1997; 65: 5198-08.
24 Yang X, Sytwn H-K, McDevitt H O, Michie S A. Involvement of β7 integrin and mucosal addressin cell adhesion molecule-1 (MAdCAM-1) in the development of diabetes in non-obese diabetic mice. Diabetes 1997; 46: 1542-7.
25 Kanwar J R, Kanwar R K, Wang D, Krissansen G W. Prevention of a chronic progressive form of experimental autoimmune encephalomyelitis by an antibody against mucosal addressin cell adhesion molecule-1, given early in the course of disease progression. Immunol. Cell. Biol. 2000; 78: 641-5.
26 Hokari R, Kato S, Matsuzaki K, Iwai A, Kawaguchi A, Nagao S, Miyahara T, Itoh K, Sekizuka E, Nagata H, Ishii H, lizuka T, Miyasaka M, Miura S. Involvement of mucosal addressin cell adhesion molecule-1 (MAdCAM-1) in the pathogenesis of granulomatous colitis in rats. Clin. Exp. Immunol. 2001; 126: 259-65.
27 Shigematsu T, Specian R D, Wolf R E, Grisham M B, Granger D N. MAdCAM mediates lymphocyte-endothelial cell adhesion in a murine model of chronic colitis. Am. J. Physiol. 2001; 281: G1309-G1315.
28 Kato S, Hokari R, Matsuzaki K, Iwai A, Kawaguchi A, Nagao S, Miyahara T, Itoh K, Ishii H, Miura S. Amelioration of murine experimental colitis by inhibition of mucosal addressin cell adhesion molecule-1. J. Pharmacol. Exp. Therap. 2000; 295: 183-89.
29 Grant A J, Lalor P F, Hubscher S G, Briskin M, Adams D H. MAdCAM-1 expressed in chronic inflammatory liver disease supports mucosal lymphocyte adhesion to hepatic endothelium (MAdCAM-1 in chronic inflammatory liver disease). Hepatology 2001; 33: 1065-72.
30 Gearing A J H, Newman W. Circulating adhesion molecules in disease. Immunol. Today 1993; 14: 506-512.
31 Lehnert K, Print C G, Yang Y, Krissansen G W. MAdCAM-1 costimulates T cell proliferation exclusively through α4β7, whereas VCAM-1 and CS-1 peptide use α4β1:evidence for “remote” costimulation and induction of hyperresponsiveness to B7 molecules. Eur. J. Immunol. 1998; 28: 3605-15.
32 Harlow D, Lane E. In: Antibodies: A Laboratory Manual. Lane D, Harlow E (eds). Cold Spring Harbor Laboratory Press, 1988.
33 Veres G, Helin T. Arato A, Farkkila M, Kantele A, Suomalainen H, Savilahti E. Increased expression of intercellular adhesion molecule-1 and mucosal adhesion molecule alpha4beta7 integrin in small intestinal mucosa of adult patients with food allergy. Clin. Immunol. 2001; 99: 353-9
34 Souza H S, Elia C C, Spencer J, MacDonald T T. Expression of lymphocyte-endothelial receptor-ligand pairs, alpha4beta7/MAdCAM-1 and OX40/OX40 ligand in the colon and jejunum of patients with inflammatory bowel disease. Gut 1999; 45: 856-63.
35 Steiniger B, Barth P, Hellinger A. The perifollicular and marginal zones of the human splenic white pulp: do fibroblasts guide lymphocyte immigration? Am. J. Pathol. 2001; 159: 501-12.
36 Ling N R. Pitfalls in the measurement of soluble forms of cell surface receptors. Clin. Exp. Immunol. 1993; 93: 139-41.
37 Hahne M, Lenter M, Jager U, vestweber D. A novel soluble form of mouse VCAM-1 is generated from a glycolipid-anchored splicing variant. Eur. J. Immunol. 1994; 24: 421-8.
38 Jackson A M, Alexandroff A B, Gribben S C, Esuvaranathan K, James K. Expression and shedding of ICAM-1 in bladder cancer and its immunotherapy. Int. J. Cancer 1993; 65: 921-25.
39 Lhotta K, Schlogl A, Kronenberg F, Joannidis M, Konig P. Soluble intercellular adhesion molecule-1 (ICAM-1) in serum and urine: correlation with renal expression of ICAM-1 in patients with kidney disease. Clin. Nephrology 1997; 48: 85-91.
40 Gbadegesin R A, Cotton S A, Coupes B M, Awan A, Brenchley P E C, Webb N J A. Plasma and urinary soluble adhesion molecule expression is increased during first documented acute pyelonephritis. Arch. Dis. Child 2002; 86: 218-21.
41 Siegel D L, “Recominant monoclonal antibody technology”, Transfus Clin Biol, 9(1):15-22 (2002).
42 Holliger P, Prospero T, and Winter G, “Diabodies: small bivalent and bispecific antibody fragments” Proc Natl Acad Sci USA, 90, 6444-6448, (1993).
43 Tomlinson I and Holliger P, “Methods for generating multivalent and bispecific antibody fragments”, Methods Enzymol, 326, 461-479, (2000).
44 “Antibodies a Laboratory Manual”, Cold Spring Harbor Laboratory Press (1988).
45 Riechmann L, Clark M, Waldmann H, Winter G. 1988. Reshaping human antibodies for therapy. Nature 332:323-27.
46 Jones P T, Dear P H, Foote J, Neuberger M S, Winter G. 1986. Replacing the complementarity determining regions in a human antibody with those from a mouse. Nature 321:522-25.
47 Wang et al, J. Immunological Methods 241: 171-184, 2000.
48 Maynard, J., and Georgiou, G. 2000. Antibody engineering. Annu Rev Biomed Eng 2: 339-376.
49 M. W. Hunkapiller, R. M. Hewick, W. J. Dreyer, and L. E. Hood. 1983. High-Sequencing with a Gas-Phase Sequenator. Methods Enzymol. 91: 399.
50 Gennaro A R: Remington: The Science and Practice of Pharmacy, 20th ed., Lippincott, Williams & Wilkins, 2000.
51 Berlin et al. 1993. α4β7 integrin mediates lymphocyte binding to the mucosal vascular addressin MAdCAM-1. Cell 74: 185-195.
52 Hatanaka et al. 2002. Increased expression of mucosal addressin cell adhesion molecule-1 and lymphocyte recruitment in murine gastritis induced by Helicobater pylori. Clin Exp Immunol Nov; 130 (2): 183-9

1-31. (canceled)

32. A monoclonal antibody having substantially the binding characteristics of an antibody designated 17F5 produced by hybridoma ATCC PTA-5010, or a fragment or derivative of said monoclonal antibody.

33. A monoclonal antibody having substantially the binding characteristics of an antibody designated 377D10 produced by hybridoma ATCC PTA-5007, or a fragment or derivative of said monoclonal antibody.

34. A monoclonal antibody having substantially the binding characteristics of an antibody designated 314G8 produced by hybridoma ATCC PTA-5008, or a fragment or derivative of said monoclonal antibody.

35. A monoclonal antibody having substantially the binding characteristics of an antibody designated 201F7 produced by hybridoma ATCC PTA-5009, or a fragment or derivative or said monoclonal antibody.

36. A monoclonal antibody having substantially the binding characteristics of an antibody designated 355G8 produced by hybridoma ATCC PTA-5011, or a fragment or derivative of said monoclonal antibody.

37. A monoclonal antibody, fragment or derivative thereof having substantially the binding characteristics of an antibody designated,
17F5 produced by hybridoma ATCC PTA-5010;
377D10 produced by hybridoma ATCC PTA-5007;
314G8 produced by hybridoma ATCC PTA-5008;
201F7 produced by hybridoma ATCC PTA-5009; or
355G8 produced by hybridoma ATCC PTA-5011; and wherein the antibody, fragment or derivative thereof binds to soluble native human MAdCAM-1.

38. The monoclonal antibody, fragment or derivative of claim 37, wherein the antibody, fragment or derivative thereof is capable of detecting MAdCAM-1 present in osteoarthritic synovium.

39. The monoclonal antibody, fragment or derivative of claim 37, wherein the antibody, fragment or derivative thereof has the isotype 1gG1 (kappa).

40. An antibody, fragment or derivative thereof, wherein the antibody, fragment or derivative thereof recognises the first 1g domain of MAdCAM-1, and has substantially the binding characteristics of an antibody designated
377D10 produced by hybridoma ATCC PTA-5007;
314G8 produced by hybridoma ATCC PTA-5008;
20G17 produced by hybridoma ATCC PTA-5009; or
355G8 produced by hybridoma ATCC PTA-5011.

41. An antibody, fragment or derivative thereof as claimed in claim 40, wherein the antibody, fragment or derivative thereof blocks the interaction of MAdCAM with the integrin α4β7.

42. An antibody, fragment or derivative thereof as claimed in claim 41, wherein the antibody, fragment or derivative blocks the interaction of MAdCAM with an α4 integrin.

43. An antibody, fragment or derivative thereof as claimed in claim 32, wherein the antibody, fragment or derivative thereof recognises the mucin domain of MAdCAM-1.

44. A monoclonal antibody selected from the group consisting of an antibody designated 17F5 produced by hybridoma ATCC PTA-5010,
an antibody designated 377D10 produced by hybridoma ATCC PTA-5007,
an antibody designated 314G8 produced by hybridoma ATCC PTA-5008,
an antibody designated 201F7 produced by hybridoma ATCC PTA-5009,
an antibody designated 355G8 produced by hybridoma ATCC PTA-5011,
or a fragment or derivative of said antibody.

45-48. (canceled)

49. A hybridoma cell line characterised by a deposit selected from the group consisting of ATCC PTA-5010, ATCC PTA-5007, ATCC PTA-5008, ATCC PTA-509, ATCC PTA-5011.

50-53. (canceled)

54. A method of blocking the interaction of MAdCAM-1 with the integrin α4β7 the method comprising at least the step of bringing said MAdCAM-1 into contact with one or more monoclonal antibody chosen from
a monoclonal antibody designated 377D10 produced by hybridoma ATCC PTA-5007,
a monoclonal antibody designated 314G8 produced by hybridoma ATCC PTA-5008,
a monoclonal antibody designated 201F7 produced by hybridoma ATCC PTA-5009,
a monoclonal antibody designated 355G8 produced by hybridoma ATCC PTA-5011,
or an antibody having substantially the binding characteristics of one of said antibodies,
or a fragment or derivative thereof or a fragment or derivative thereof.

55. The method of claim 54 wherein the method is for blocking leukocyte adhesion to MAdCAM-1.

56. A method of detecting the presence of MAdCAM-1 in a sample, the method comprising at least the steps of:
a. placing the sample in contact with one or more of the monoclonal antibodies chosen from
a monoclonal antibody designated 17F5 produced by hybridoma ATCC PTA-5010,
a monoclonal antibody designated 377D10 produced by hybridoma ATCC PTA-5007,
a monoclonal antibody designated 314G8 produced by hybridoma ATCC PTA-5008,
a monoclonal antibody designated 201F7 produced by hybridoma ATCC PTA-5009,
a monoclonal antibody designated 355G8 produced by hybridoma ATCC PTA-5011, or
an antibody having substantially the binding characteristics of one of said antibodies or a fragment or derivative thereof
for sufficient time to allow binding between the one or more antibodies or fragments or derivatives thereof and MAdCAM-1 if present in the sample; and,
b. detecting whether or not binding has occurred;
wherein binding is indicative of the presence of MAdCAM-1.

57. A method of diagnosing or monitoring inflammation in a subject, the method comprising at least the steps of:
a. placing the sample in contact with one or more of the monoclonal antibodies chosen from
a monoclonal antibody designated 17F5 produced by hybridoma ATCC PTA-5010
a monoclonal antibody designated 377D10 produced by hybridoma ATCC PTA-5007,
a monoclonal antibody designated 314G8 produced by hybridoma ATCC PTA-5008,
a monoclonal antibody designated 201F7 produced by hybridoma ATCC PTA-5009,
a monoclonal antibody designated 355G8 produced by hybridoma ATCC PTA-5011, or
an antibody having substantially the binding characteristics of one of said antibodies, or a fragment or derivative thereof
for sufficient time to allow binding between the one or more antibodies or fragments or derivatives thereof with MAdCAM-1 if present in the sample;
b. detecting the level of binding, wherein the level of binding is indicative of the level of MAdCAM-1 present in the sample;
c. comparing the level of MAdCAM-1 present in the sample to a base level or standard.

58. The method of claim 57 wherein a statistically significant increase in the level of MAdCAM-1 above the base level or standard is diagnostic of inflammation.

59. The method of in claim 56 or 57 wherein the sample is a tissue or fluid sample.

60. The method of claim 59 wherein the fluid sample is serum, plasma or urine.

61. The method of claim 56 or 57 wherein the method involves one or more of:
ELISA;
RIA;
Immunoprecipitation;
Western blotting;
Immunohistochemical staining;
Affinity Chromatography;
Competitive binding assays; and
Agglutination assays.

62. The method of claim 61 wherein the method involves ELISA and the ELISA is a sandwich ELISA utilising a capture antibody and a detection antibody, wherein:
a) the capture antibody is chosen from a monoclonal antibody designated 17F5 produced by hybridoma ATCC PTA-5010, a monoclonal antibody designated 355G8 produced by hybridoma ATCC PTA-5011, or an antibody having substantially the binding characteristics of one of said antibodies, or fragments or derivatives thereof; and,
b) the detection antibody is chosen from
a monoclonal antibody designated 377D10 produced by hybridoma ATCC PTA-5007,
a monoclonal antibody designated 314G8 produced by hybridoma ATCC PTA-5008,
a monoclonal antibody designated 201F7 produced by hybridoma ATCC PTA-5009,
or an antibody having substantially the binding characteristics of one of said antibodies,
or a fragment or derivative thereof.

63. The method as claimed in claim 61 wherein the method involves ELISA and the ELISA is a sandwich ELISA utilising a capture antibody and a detection antibody, wherein: a) the capture antibody is chosen from
a monoclonal antibody designated 377D10 produced by hybridoma ATCC PTA-5007,
a monoclonal antibody designated 314G8 produced by hybridoma ATCC PTA-5008,
a monoclonal antibody designated 201F7 produced by hybridoma ATCC PTA-5009,
or an antibody having substantially the binding characteristics of one of said antibodies
or fragment or derivative thereof; and,
b) the detection antibody is chosen from
a monoclonal antibody designated 17F5 produced by hybridoma ATCC PTA-5010, an antibody a monoclonal antibody designated 355G8 produced by hybridoma ATCC PTA-5011, or an antibody having substantially the binding characteristics of one of said antibodies, or fragments or derivatives thereof.

64. A method as claimed in any one of claims 54, 56 or 57 wherein the MAdCAM-1 is soluble MAdCAM-1.

65. A method as claimed in any one of claims 54, 56 or 57 wherein the MAdCAM-1 is membrane-bound MAdCAM-1.

66. A method as claimed in claim 56 or 57 wherein the method is for diagnosing or monitoring one or more of the following inflammatory disorders: appendicitis, pancreatitis, inflammatory bowel disease including Crohn's disease and ulcerative colitis, inflammatory liver disease, diabetes, multiple sclerosis and other demyelinating diseases, duodenal ulcers, arthritis, asthma and other allergic inflammatory diseases, atherosclerosis, and inflammation associated with transplantation and chronic infection.

67. A method as claimed in claim 66 wherein the method is for diagnosing or monitoring Crohn's disease.

68. A method as claimed in claim 66 wherein the method is for diagnosing or monitoring osteoarthritis.

69. A pharmaceutical composition comprising at least one or more of the a monoclonal antibody selected from the group consisting of
a monoclonal antibody designated 377D10 produced by hybridoma ATCC PTA-5007;
a monoclonal antibody designated 314G8 produced by hybridoma ATCC PTA-5008;
a monoclonal antibody designated 201F7 produced by hybridoma ATCC PTA-5009; and
a monoclonal antibody designated 355G8 produced by hybridoma ATCC PTA-5011; or
an antibody having substantially the binding characteristics of one or said antibodies,
or a fragment or derivative thereof in combination with one or more pharmaceutically acceptable diluents, carriers and/or excipients.

70. A method for the treatment of inflammation in a subject the method comprising at least the step of administering to said subject the pharmaceutical composition of claim 69.

71. A method as claimed in claim 70, wherein the pharmaceutical composition is for the treatment of one or more of the following inflammatory disorders: appendicitis, pancreatitis, inflammatory bowel disease including Crohn's disease and ulcerative colitis, inflammatory liver disease, diabetes, multiple sclerosis and other demyclinating diseases, duodenal ulcers, arthritis, asthma and other allergic inflammatory diseases, atherosclerosis, and inflammation associated with transplantation and chronic infection.

72. A method as claimed in claim 71, wherein the pharmaceutical composition is for the treatment of Crohn's disease.

73. A method as claimed in claim 71, wherein the pharmaceutical composition is for the treatment of osteoarthritis.

74. A kit for detecting MAdCAM-1 in a sample or diagnosing or monitoring inflammation in a subject, the kit comprising at least one or more of the monoclonal antibodies or
a monoclonal antibody designated 17F5 produced by hybridoma ATCC PTA-5010,
a monoclonal antibody designated 377D10 produced by hybridoma ATCC PTA-5007,
a monoclonal antibody designated 314G8 produced by hybridoma ATCC PTA-5008,
a monoclonal antibody designated 201F7 produced by hybridoma ATCC PTA-5009,
a monoclonal antibody designated 355G8 produced by hybridoma ATCC PTA-5011, or
an antibody having substantially the binding characteristics of one of said antibodies or a fragment or derivative thereof.

75. A kit as claimed in claim 74, wherein it includes a capture antibody and a detection antibody, wherein:
a) the capture antibody is chosen from a monoclonal antibody designated 17F5 produced by hybridoma ATCC PTA-5010,
a monoclonal antibody designated 355G8 produced by hybridoma ATCC PTA-5011,
or an antibody having substantially the binding characteristics of one of said antibodies; and,
b) the detection antibody is chosen from
a monoclonal antibody designated 377D10 produced by hybridoma ATCC PTA-5007;
a monoclonal antibody designated 314G8 produced by hybridoma ATCC PTA-5008;
monoclonal antibody designated 201F7 produced by hybridoma ATCC PTA-5009; and
a monoclonal antibody designated 355G8 produced by hybridoma ATCC PTA-5011;
or an antibody having substantially the binding characteristics of one or more of said antibodies,
a fragment or derivative thereof.

76. A kit as claimed in claim 74 wherein it includes a capture antibody and a detection antibody, wherein: a) the capture antibody is chosen from
a monoclonal antibody designated 377D10 produced by hybridoma ATCC PTA-5007;
a monoclonal antibody designated 314G8 produced by hybridoma ATCC PTA-5008;
a monoclonal antibody designated 201F7 produced by hybridoma ATCC PTA-5009; and
a monoclonal antibody designated 355G8 produced by hybridoma ATCC PTA-5011;
or an antibody having substantially the binding characteristics of one or more of said antibodies; and, b) the detection antibody is chosen from
a monoclonal antibody designated 17F5 produced by hybridoma ATCC PTA-5010,
a monoclonal antibody designated 355G8 produced by hybridoma ATCC PTA-5011;
or an antibody having substantially the binding characteristics of one or more of said antibodies or fragment or derivative thereof.

77. A kit as claimed in claim 74, wherein the kit further comprises one or more control samples comprising a known level of MAdCAM-1 or a fragment or derivative thereof.
